(19) United States
(12) Patent Application Publication
(10) Pub. No.: US 2016/0137573 A1
(43) Pub. Date: May 19, 2016

(54) METHODS AND CATALYST SYSTEMS FOR CARBON DIOXIDE CONVERSION

(71) Applicant: Hongfei Lin, Reno, NV (US)

(72) Inventor: Hongfei Lin, Reno, NV (US)

(73) Assignee: Board of Regents of the Nevada System of Higher Education, on Behalf of the University of Nevada,, Reno, NV (US)

(21) Appl. No.: 14/942,910

(22) Filed: Nov. 16, 2015

Related U.S. Application Data
(60) Provisional application No. 62/080,109, filed on Nov. 14, 2014.

(51) Int. Cl. C07C 51/00

(52) U.S. Cl. CPC C07C 51/00

(57) ABSTRACT

Disclosed herein are embodiments of a heterogeneous catalyst system and methods of using the same to convert CO₂-derived compounds to formate, formic acid, or a mixture thereof. The disclosed heterogeneous catalyst systems exhibit superior reactivity and stability in comparison to homogeneous catalyst systems and also can convert a variety of CO₂-derived compounds to formate, formic acid, or mixtures thereof, in high yields using economical and environmentally friendly reaction conditions.

### CROSS REFERENCE TO RELATED APPLICATION

This application claims the benefit of U.S. Provisional Patent Application No. 62/080,109, filed on Nov. 14, 2014, the entirety of which is incorporated herein by reference.

### FIELD

The present disclosure concerns embodiments of methods and catalyst systems for forming energetic substances, such as formate, formic acid, or mixtures thereof, from carbon dioxide.

### BACKGROUND

Creating a sustainable supply of energy is one of the central challenges of the 21^{st} century. Hydrogen is the cleanest energy carrier, and thus methods of advancing hydrogen technologies are of particular interest in the field. Hydrogen technologies can include generating hydrogen from renewable materials, hydrogen storage, and converting hydrogen into electrical energy. Also, catalytic conversion of carbon dioxide to fuels or energy sources (e.g., methane, methanol, formaldehyde, formic acid, and organic carbonates) can be used not only to obtain fuel sources, like hydrogen gas, but also to reduce the amount of carbon dioxide (CO₂) released into the atmosphere from combusting fossil fuels.

Formate, formic acid, or mixtures thereof, if obtained from CO₂ hydrogenation, can be a promising source for carbon-neutral hydrogen storage. Presently, however, there are no efficient CO₂ hydrogenation processes used in the art to produce formic acid without the addition of additives, such as organic amines or inorganic bases. With base additives, formic acid is converted to formate salts, which are non-corrosive, nonirritating, and easy to handle, as well as highly soluble in water. Base additives also can be used to catalyze the hydration of CO₂ to form bicarbonate species in water. Therefore, the bicarbonate/formate equilibrium in aqueous solutions [Equation. (1)] can be used for hydrogen storage and evolution.

HCO₃⁻+H₂ [symbol] HCO₂⁻+H₂O (1)

A need exists in the art, however, for economically feasible and efficient methods to convert carbon dioxide to useful energy sources.

### SUMMARY

Disclosed herein are embodiments of a method for producing formate(s), formic acid, or a mixture thereof, from CO₂, comprising exposing a CO₂-derived compound to a heterogeneous catalyst system comprising palladium (Pd) and a carbon-based material and also exposing the CO₂-derived compound to H₂ gas at a pressure ranging from 300 psi to 500 psi. In some embodiments, the CO₂-derived compound is exposed to the heterogeneous catalyst system and the H₂ gas at a temperature and for a time suitable to produce formate(s), formic acid, or a mixture thereof. In some embodiments, the method can further comprise exposing CO₂ to an amine-containing compound to form the CO₂-derived compound.

Amine-containing compounds disclosed herein can be selected from ammonia, or a compound having a formula NH₂R^{a}, wherein R^{a} is aliphatic or heteroaliphatic; NH(R^{a})₂, wherein each R^{a} independently is aliphatic, heteroaliphatic, or wherein both R^{a} groups form, together with the nitrogen atom to which they are attached, a heterocyclic group comprising from 0 to 3 additional heteroatoms in addition to the nitrogen atom to which each R^{a} is attached; or N(R^{a})₃, wherein each R^{a} independently is aliphatic, heteroaliphatic, or wherein two or three R^{a} groups form, together with the nitrogen atom to which they are attached, a heterocyclic group comprising from 0 to 3 additional heteroatoms in addition to the nitrogen atom to which each R^{a} is attached. In some embodiments, the amine-containing compound can be selected from ammonia, monoethanolamine, diethanolamine, triethanolamine, 2-amino-2-methyl-1-propanol, N-methyldiethanolamine, N-methylethanolamine, 1,4-diaminobutane, 1,3-diamino-2-propanol, 2-(diethylamino)ethanol, 1,3-propanediamine, 2-diisopropylamino-ethanol, 2,2-dimethyl-1,3-propanediamine, N-1-methyl-1,3-propanediamine, N-tert-butyldiethanolamine, piperazine, piperidine, pyrrolidine, homopiperazine, 1-piperazineethanol, or combinations thereof. In exemplary embodiments, the amine-containing compound is 2-amino-2-methyl-1-propanol or ammonia.

In some embodiments, the CO₂-derived compound is in an aqueous solution. The aqueous solution also can comprise an alcohol co-solvent, such as an alcohol or an organic solvent. In some embodiments, the aqueous solution comprises a solvent system comprising water and 20 wt % to 90 wt % ethanol. In yet additional embodiments, the solvent system comprises water and 95.6 wt % ethanol.

The heterogeneous catalyst system typically comprises Pd nanoparticles supported on activated carbon. In some embodiments, the CO₂-derived compound is exposed to H₂ at a pressure ranging from 350 psi to 450 psi. The temperature used for the method can range from 20° C. to 80° C. In some embodiments, the CO₂-derived compound is exposed to the heterogeneous catalyst system and H₂ for a time period ranging from 20 minutes to 6 hours.

In some embodiments, the CO₂-derived compound has a formula (Z)₂CO₃, wherein each Z independently is selected from a metal, hydrogen, ammonium, or a quaternary ammonium group. In such embodiments, the CO₂-derived compound can be selected from a metal bicarbonate, ammonium bicarbonate, a metal carbonate, ammonium carbonate, or a combination thereof. In some embodiments, the CO₂-derived compound can be selected from a bicarbonate other than sodium bicarbonate, a carbonate, a carbamate, or a combination thereof. In some embodiments, the CO₂-derived compound is selected from potassium bicarbonate, ammonium bicarbonate, potassium carbonate, sodium carbonate, magnesium carbonate, calcium carbonate, ammonium carbonate, or a combination thereof. In exemplary embodiments, the CO₂-derived compound is ammonium bicarbonate, ammonium carbamate, (1-hydroxy-2-methylpropan-2-yl)carbamate, or a combination thereof. In one exemplary embodiment the CO₂-derived compound is first converted to a bicarbonate other than sodium bicarbonate and then to formate, formic acid, or a mixture thereof. In another exemplary embodiment, the CO₂-derived compound is converted to a carbamate and then to formate, formic acid, or a mixture thereof. In yet another exemplary embodiment, the CO₂-derived compound is converted directly to formate, formic acid, or a mixture thereof.

Also disclosed herein is a method of converting CO₂ to formate, formic acid, or a mixture thereof, comprising exposing CO₂ to an amine-containing compound to form a carbamate; exposing the carbamate to a heterogeneous catalyst system comprising Pd and a carbon-based material; and exposing the carbamate to H₂ gas at a pressure ranging from 300 psi to 500 psi; wherein the carbamate is exposed to the heterogeneous catalyst system and the H₂ gas at a temperature and for a time suitable to produce formate, formic acid, or a mixture thereof.

Other embodiments concern a method of converting CO₂ to formate, formic acid, or a mixture thereof, comprising exposing ammonium bicarbonate to a heterogeneous catalyst system comprising Pd and a carbon-based material; and exposing the ammonium bicarbonate to H₂ gas at a pressure ranging from 300 psi to 500 psi; wherein the ammonium bicarbonate is exposed to the heterogeneous catalyst system and the H₂ gas at a temperature and for a time suitable to produce formate, formic acid, or a mixture thereof.

Also disclosed herein are embodiments of a combination, comprising ammonium bicarbonate or a carbamate and a heterogeneous catalyst system comprising Pd and a carbon-based material. In some embodiments, the combination further comprises H₂ gas. In other embodiments, the combination further comprises an aqueous solvent, which also can further comprise an alcohol co-solvent, such as ethanol. In particular disclosed embodiments, the carbamate is (1-hydroxy-2-methylpropan-2-yl)carbamate or ammonium carbamate.

The foregoing and other features and advantages of the present disclosure will become more apparent from the following detailed description, which proceeds with reference to the accompanying figures.

### BRIEF DESCRIPTION OF THE DRAWINGS

FIG. 1 is a schematic diagram of a representative formate decomposition reaction system.

FIG. 2 is a TEM image of a Pd/AC catalyst.

FIG. 3 is a TEM image of a Pd/Al₂O₃ catalyst.

FIG. 4 is a TEM image of a Ru/AC catalyst.

FIG. 5 is a TEM image of an Rh/AC catalyst

FIG. 6 is a TEM image of a fresh Pd/AC catalyst system before 1 cycle of ammonium bicarbonate hydrogenation and then ammonium formate dehydrogenation.

FIG. 7 is a Pd nanoparticle size distribution graph of the Pd/AC catalyst system of FIG. 6.

FIG. 8 is a TEM image of the Pd/AC catalyst system of FIG. 6 after 1 cycle of ammonium bicarbonate hydrogenation and then ammonium formate dehydrogenation.

FIG. 9 is a Pd nanoparticle size distribution graph of the Pd/AC catalyst system of FIG. 8.

FIG. 10 is a graph of formate yield (%) as a function of reaction time (hours) illustrating the temperature effect on the hydrogenation of NH₄HCO₃.

FIG. 11 is a combined ¹³C-NMR spectrum illustrating the ¹³C-NMR spectra of various bicarbonate salts in water.

FIG. 12 is a graph of hydrogen yield (%) as a function of time (minutes) illustrating reaction temperature effect on the dehydrogenation of NH₄HCO₂ and NaHCO₂.

FIG. 13 is a graph of intensity as a function of 2 theta illustrating the XRD patterns of the Pd/AC catalysts of FIGS. 6 (bottom) and 8 (top).

FIG. 14 is a graph of intensity as a function of binding energy (ev) illustrating the Pd 3-dimensional atomic orbital XPS spectra of different Pd/AC catalyst system samples, particularly Pd on activated charcoal before reaction (top) and Pd on activated carbon after 1 cycle of ammonium bicarbonate hydrogenation and then ammonium formate dehydrogenation (bottom).

FIG. 15 is a gas chromatogram illustrating the gaseous products obtained after NH₄CO₂H decomposition.

FIG. 16 is a graph of formate yield (%) as a function of reaction time (hour) illustrating the effect of reaction temperature and time on the production of formate using AMP as a capture agent.

FIG. 17 is a bar graph of turnover number versus catalyst usage times illustrating the results obtained from stability testing of Pd/AC catalyst system in captured CO₂ hydrogenation.

FIG. 18 is a graph of formate yield as a function of H₂ pressure (psi).

FIG. 19 is graph of capture capacity (mol CO₂/mol amine) as a function of capture time (minutes) illustrating the absorption rate of CO₂ in an amine/water solution using monoethanolamine (MEA), diethanolamine (DEA) and Triethanolamine (TEA), 2-amino-2-methyl-1-propanol (AMP) and Piperazine (PZ).

FIG. 20 is a combined ¹³C-NMR spectrum illustrating the ¹³C-NMR spectra of CO₂ captured by MEA, DEA, TEA, PZ and AMP in water.

FIG. 21 is graph of formate yield (%) as a function of wt % of organic solvent illustrating the effect of an organic co-solvent on formate production.

FIG. 22 is graph of capture capacity (mol CO₂/mol amine) as a function of capture time (minutes) illustrating the effect of co-solvent on the CO₂ capture rate with AMP.

FIG. 23 is a graph of formate yield (%) as a function of solvent (wt % ethanol in water) illustrating the effect of an ethanol-water co-solvent system on the hydrogenation efficiency of amine-captured CO₂.

FIG. 24 is a combined ¹³C-NMR spectrum illustrating the effect of a co-solvent system on the distribution of carbonate, bicarbonate, and carbamate.

FIG. 25 is an HPLC spectrum of the product distribution obtained after an ammonium carbamate hydrogenation reaction.

FIG. 26 is graph of formate yield (%) as a function of reaction time (hours) illustrating the temperature effect on the hydrogenation of ammonium carbamate.

FIG. 27 is a graph of formate yield (%) as a function of solvent system (ethanol/water, wt %) illustrating the solvent effect on the hydrogenation of different carbon dioxide derived salts.

FIG. 28 illustrates three different combined ¹³C-NMR spectra illustrating ¹³C-NMR spectra of NH₂CO₂NH₄, (NH₄)₂CO₃, NaHCO₃, and Na₂CO₃ in 100 wt % ethanol (left), 100 wt % water (middle), and 70 wt % ethanol (right).

FIG. 29 is a bar graph of formate yield (%) versus times of usage, illustrating the results obtained from stability tests for the Pd/AC catalyst system during an ammonium carbamate hydrogenation reaction.

FIG. 30 is a combined ¹³C-NMR spectrum illustrating the ¹³C-NMR spectra of a reaction solution before and after an ammonium carbamate hydrogenation reaction.

FIGS. 31A and 31B are combined ¹³C NMR spectra illustrating results obtained from evaluating the effect of co-solvent on the distribution of carbonate, bicarbonate and carbamate (0%-100% wt % ETOH 20 ml, amine 1 M, capture temperature 20° C.).

FIG. 32 is a combined ¹³C NMR spectrum illustrating results obtained from evaluating the effect of co-solvent (methanol and water) on the distribution of carbonate, bicarbonate, and carbamate.

FIG. 33 is a graph of turn over number (TON) as a function of reaction time (hours) illustrating the hydrogenation of AMP captured CO₂ with ethanol (100 wt %) as solvent.

FIG. 34 is a graph of formate yield (%) as a function of organic solvent weight percent (wt %) illustrating the effect of organic co-solvent, wherein the capture agent was AMP and the CO₂ capture capacity was 0.96.

FIGS. 35A and 35B are combined ¹³C NMR spectra illustrating results obtained from evaluating the effect of reaction time on the distribution of intermediates and products in a hydrogenation reaction.

FIG. 36 is a combined ¹³C NMR spectrum illustrating spectra obtained from evaluating AMP in a pure ethanol solvent after CO₂ capture and a hydrogenation reaction.

FIGS. 37A and 37B illustrate results obtained from representative embodiments disclosed herein; FIG. 37A is a graph of formate concentration (M) as a function of reaction time (minutes) illustrating the formate concentration in the hydrogenation of AMP-captured CO₂ wherein the concentration profile was fitted into first order reaction kinetics (C_{formate}=C₀−C₀e^{(-Kt)}; FIG. 37B is a graph of initial reaction rate of CO₂ hydrogenation (Ro) at t=0, plotted as function of initial concentration of carbon dioxide.

FIG. 38 is a graph illustrating the first-order rates for hydrogenation of bicarbonate and ethyl carbonate at different temperatures.

FIG. 39 is a graph of formate yield (%) as a function of ethanol weight percent (wt %) illustrating the effect of ethanol-water co-solvent on hydrogenation efficiency of the amine-captured CO₂ for representative embodiments disclosed herein.

FIG. 40 is an x-ray diffraction (XRD) pattern of a Pd/AC catalyst system before (“fresh catalyst”) and after (“spent catalyst”) five hydrogenation reaction cycles.

FIG. 41 is a combined ¹³C NMR spectrum illustrating the ¹³C NMR spectra of a CO₂-piperidine-50% ETOH reaction mixture before (top) and after (bottom) performing a hydrogenation reaction.

### DETAILED DESCRIPTION

### I. Explanation of Terms

The following explanations of terms are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements, unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting, unless otherwise indicated. Other features of the disclosure are apparent from the following detailed description and the claims.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under standard test conditions/methods. When directly and explicitly distinguishing embodiments from discussed prior art, the embodiment numbers are not approximates unless the word “about” is recited. Furthermore, not all alternatives recited herein are equivalents.

Aliphatic: A hydrocarbon, or a radical thereof, having at least one carbon atom to 50 carbon atoms, such as one to 25 carbon atoms, or one to ten carbon atoms, and which includes alkanes (or alkyl), alkenes (or alkenyl), alkynes (or alkynyl), including cyclic versions thereof, and further including straight- and branched-chain arrangements, and all stereo and position isomers as well.

Alkenyl: An unsaturated monovalent hydrocarbon having at least two carbon atoms to 50 carbon atoms, such as two to 25 carbon atoms, or two to ten carbon atoms, and at least one carbon-carbon double bond, wherein the unsaturated monovalent hydrocarbon can be derived from removing one hydrogen atom from one carbon atom of a parent alkene. An alkenyl group can be branched, straight-chain, cyclic (e.g., cylcoalkenyl), cis, or trans (e.g., E or Z).

Alkoxy: —O-alkyl, —O-alkenyl, or —O-alkynyl, with exemplary embodiments including, but not limited to, methoxy, ethoxy, n-propoxy, isopropoxy, n-butoxy, t-butoxy, sec-butoxy, n-pentoxy.

Alkyl: A saturated monovalent hydrocarbon having at least one carbon atom to 50 carbon atoms, such as one to 25 carbon atoms, or one to ten carbon atoms, wherein the saturated monovalent hydrocarbon can be derived from removing one hydrogen atom from one carbon atom of a parent compound (e.g., alkane). An alkyl group can be branched, straight-chain, or cyclic (e.g., cycloalkyl).

Alkynyl: An unsaturated monovalent hydrocarbon having at least two carbon atoms to 50 carbon atoms, such as two to 25 carbon atoms, or two to ten carbon atoms and at least one carbon-carbon triple bond, wherein the unsaturated monovalent hydrocarbon can be derived from removing one hydrogen atom from one carbon atom of a parent alkyne. An alkynyl group can be branched, straight-chain, or cyclic (e.g., cycloalkynyl).

Amine-Containing Compound: A compound, typically an organic compound, comprising at least one nitrogen atom (e.g., ammonia or a compound comprising one or more primary, secondary, or tertiary amine groups) and that is capable of reacting with CO₂ to form a carbamate species.

Carbamate: A compound having a formula H₂NC(O)OX, HR^{a}NC(O)OX, or (R^{a})₂NC(O)O^{a}X, wherein X is a counter ion electrostatically or ionically bound to or associated with O^{a}.

Carbon-based material: A carbon-based material is a material comprising, consisting of, or consisting essentially of carbon atoms. In particular disclosed embodiments, a carbon-based material can be selected from activated carbon materials, graphite, graphene, carbon black, carbon fibers, carbon nanomaterials, and the like. In yet additional embodiments, a carbon-based material can be a material comprising, consisting of, or consisting essentially of carbon atoms and a dopant, such as a dopant selected from nitrogen, boron, oxygen, phosphorous, aluminum, gallium, indium, or combinations thereof.

Formate(s): A compound having a formula HC(O)O⁻, or HC(O)O⁻X, wherein X is a counter ion electrostatically or ionically bound to or associated with the negatively charged oxygen atom of the formula HC(O)O⁻.

Heteroaliphatic: An aliphatic group (which can be branched, straight-chain, or cyclic) comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heteroalkyl/Heteroalkenyl/Heteroalkynyl: An alkyl, alkenyl, or alkynyl group (which can be branched, straight-chain, or cyclic) comprising at least one heteroatom to 20 heteroatoms, such as one to 15 heteroatoms, or one to 5 heteroatoms, which can be selected from, but not limited to oxygen, nitrogen, sulfur, selenium, phosphorous, and oxidized forms thereof within the group.

Heterogeneous Catalyst System: A catalyst system that is present in a different phase from that of the reactants and products of a particular reaction. In particular disclosed embodiments, a heterogeneous catalyst system is a solid and can comprise two different components. Solely by way of example, heterogeneous catalysts contemplated by the present disclosure can include, but are not limited to, catalysts comprising a metal and a support material, wherein the metal is selected from Pd, Ru, Rh, Pt, or Ni and the support material is selected from a carbon-based material, metal organic frameworks, covalent organic frameworks, metal oxides, metal carbonates, or metal sulfates. The heterogeneous catalyst systems disclosed herein are distinct from and do not include pseudo-homogeneous catalyst systems, such as homogeneous catalyst immobilized on a support.

Nanoparticle: A nanoscale particle with a size that is measured in nanometers, for example, a nanoscopic particle that has at least one dimension of less than about 100 nm.

Primary Amine: NH₂R^{a}, wherein R^{a} is aliphatic, such as alkyl, alkenyl, or alkynyl, or heteroaliphatic, such as an aliphatic group substituted with a hydroxyl, a thiol, an alkoxy, a thioether, or an amine group.

Secondary Amine: NH(R^{a})₂, wherein each R^{a} independently is aliphatic, such as alkyl, alkenyl, or alkynyl, heteroaliphatic, such as an aliphatic group substituted with a hydroxyl, a thiol, an alkoxy, a thioether, or an amine group, or wherein both R^{a} groups form, together with the nitrogen atom to which they are attached, a heterocyclic group comprising from 0 to 3 additional heteroatoms (in addition to the nitrogen atom to which each R^{a} is attached).

Tertiary Amine: N(R^{a})₃, wherein each R^{a} independently is aliphatic, such as alkyl, alkenyl, or alkynyl, heteroaliphatic, such as an aliphatic group substituted with a hydroxyl, a thiol, an alkoxy, a thioether, or an amine group, or wherein two or three R^{a} groups form, together with the nitrogen atom to which they are attached, a heterocyclic group comprising from 0 to 3 additional heteroatoms (in addition to the nitrogen atom to which each R^{a} is attached).

Thioether: —S-alkyl, —S-alkenyl, or —S-alkynyl, with exemplary embodiments including, but not limited to, —SCH₃, —SCH₂CH₃, —SCH₂CH₂CH₃, —SiCH₂CH₂CH₃, —SCH₂CH₂CH₂CH₃, —SCH₂CH₂CH₂CH₂CH₃.

### II. Overview

Many difficulties can be associated with converting carbon dioxide to fuels and/or energy sources. Also, direct hydrogenation of CO₂ in the gas-phase is thermodynamically unfavorable and often requires high external energy input. Traditional methods used to overcome these difficulties have included using base additives, such as ammonia or amines, in an aqueous phase to convert CO₂ to soluble bicarbonate salts. These methods, however, rely on the use of homogeneous catalysis to produce desired results. Due to reliance on homogeneous catalysts, these methods often are not scalable or cost effective and therefore not applicable in industry.

Traditional methods used to reduce sodium bicarbonate salts to formate salts using hydrogen gas and a Pd/carbon catalyst systems suffer from the inability to efficiently convert the sodium bicarbonate to formate. For example, these methods do not produce scalable amounts of formate and require high catalyst loadings. Additionally, such methods require using homogeneous catalysts to improve reaction efficiency. Homogeneous catalysts, however, are harder to separate from reaction products and are not as stable as heterogeneous catalysts. Additionally, homogeneous catalysts add to the inefficient complexity of these methods as they often cannot be separated, reused, deactivated, and/or regenerated. Traditional methods of generating formate from bicarbonate also rely on using sodium bicarbonate. This particular reagent, however, exhibits low solubility in water, which negatively impacts the yield of formate(s) produced from using these conventional methods. Accordingly, methods for generating formate(s) from bicarbonate species typically utilize homogeneous catalysts to address this issue.

The methods and catalyst systems disclosed herein overcome many of these deficiencies associated with conventional methods for carbon dioxide conversion to fuels, such as formate. The methods and catalyst systems disclosed herein can be used in an aqueous phase, rather than gas phase, thereby providing a thermodynamically favorable reaction to produce a formate from CO₂. Additionally, the methods disclosed herein can be used to convert amine-captured CO₂ to formate(s) or formic acid directly, without requiring the separation, compression, and transportation techniques currently used in the art for carbon capture and storage from power plants, which can be expensive. These methods also can be used to leverage industrial urea production.

### III. Catalyst Systems

Disclosed herein are embodiments of catalyst systems that can be used to covert carbon dioxide to energy sources, such as formate, formic acid, or mixtures thereof. In particular disclosed embodiments, the catalyst systems are heterogeneous and can be used to convert CO₂-derived compounds into formate, formic acid, or mixtures thereof. In some disclosed embodiments, the catalysts can include metal nano-cluster catalysts suitable for use in aqueous media or co-solvent systems. In some embodiments, the catalysts comprise a metal and a support material.

The metal of the catalyst system can be selected from a Group 8 metal, a Group 9 metal, or a Group 10 metal. In particular disclosed embodiments, the metal is selected from Pd, Ru, Rh, Pt, or Ni. In exemplary embodiments, the metal is palladium. The support material can be a hydrophilic or hydrophobic support material. In particular disclosed embodiments, the support material is capable of attracting, or storing H₂, formed during the reaction process described herein. Without being limited to a single theory of operation, it is currently believed that the ability of the support material to localize H₂ on the catalysts system can promote a higher yield of formate(s) from CO₂, however, embodiments disclosed herein are not solely limited to any such particular support materials. In some embodiments, the support materials can be selected from activated carbon materials, graphene, metal organic frameworks, covalent organic frameworks, metal oxides (e.g., aluminum oxide), metal carbonates (e.g., calcium carbonates), or metal sulfates (e.g., barium sulfate). In exemplary embodiments, the catalyst system is a mixed system of Pd and activated carbon. In some embodiments, the catalyst system is a mixed system of Pd nanoparticles and activated carbon. In an independent embodiment, the catalyst system is a heterogeneous system that consists of or consists essentially of Pd nanoparticles and activated carbon. In such embodiments wherein the catalyst system consists essentially of Pd nanoparticles and activated carbon, the system is free of inorganic additives, such as inorganic bases selected from sodium hydroxide, potassium hydroxide, and the like. Such inorganic bases form intermediates with CO₂ that require high temperatures to degrade the intermediates. Accordingly, the inorganic bases cannot be recycled and reused to capture more CO₂, unlike the present amine-containing compounds discussed in more detail herein. In certain disclosed embodiments, the catalysts can be obtained from commercial sources.

The catalyst systems disclosed herein can be used in any suitable amount for converting CO₂ to formate. In particular disclosed embodiments, the catalyst system is provided in a catalytic amount rather than stoichiometric amounts. For example, the catalyst system can be provided at a 0.01% to 100% catalyst loading, such as 0.5% to 50% catalyst loading, or 1% to 10% catalyst loading (wherein the % catalyst loading is based on the CO₂ species being converted to formate). In independent embodiments, the amount of the catalyst system that is used is not, or is other than, 5 g Pd catalyst (10 wt %) per 100 ml of a sodium bicarbonate solution.

In some embodiments, the catalyst system is capable of producing formate(s) in yields ranging from 10% to 100%, such as 30% to 100%, or 70% to 100%, such as 70%, 75%, 80%, 85%, 90%, 95%. In exemplary embodiments, the catalyst system embodiments disclosed herein are capable of producing unexpectedly superior formate(s) yields in comparison to homogenous catalyst systems used in the art, such as ruthenium-containing catalysts, (e.g., [{RuCl₂(benzene)}₂]). In an exemplary embodiment, the disclosed heterogeneous catalyst system produced a 90% yield of formate(s), whereas a homogeneous catalyst, [{RuCl₂(benzene)}₂], produced only a 35% yield of formate(s) under similar reaction conditions.

In addition to providing superior yields of formate(s) from CO₂, the heterogeneous catalyst systems disclosed herein exhibit superior stability as compared to conventional homogenous catalysts. For example, heterogeneous catalyst systems comprising Pd nanoparticles disclosed herein do not exhibit sintering or aggregation and can be used in repetitive cycles of the reactions disclosed herein without exhibiting reduced catalytic activity. Methods known to those of ordinary skill in the art, such as x-ray diffraction analysis, can be used to evaluate the integrity of the catalyst. In particular disclosed embodiments, the heterogeneous catalyst systems can be used in any number of reaction cycles. In some embodiments, the catalyst systems described herein can be used to form a hydrogen battery, wherein the catalyst system is used in combination with formate(s) species disclosed herein (e.g., ammonium formate) to evolve H₂, which can then be used as an energy source.

In some embodiments, the support material of the catalyst system can be doped. In such embodiments, the dopant can be added to the support material to influence the electrical and/or chemical properties of the support material and/or catalyst system. Suitable dopants include, but are not limited to nitrogen, boron, oxygen, phosphorus, aluminum, phosphorus, tin, gallium, nickel, indium, and combinations thereof. In yet additional embodiments, the metal component of the catalyst system can be alloyed. In such embodiments, the metal component used in combination with a support material can be alloyed with one or more additional metals to form an alloyed catalyst system. Suitable metals for use in forming an alloyed catalyst system include transition metals, such as, but not limited to, gold (Au), platinum (Pt), rhodium (Rh), ruthenium (Ru), iridium (Ir), and osmium (Os).

### IV. Methods

Also disclosed herein are embodiments of a method for converting a CO₂-derived compound to formic acid, formate(s), or a mixture thereof. In particular disclosed embodiments, the method can comprise exposing a CO₂-derived compound to a heterogeneous catalyst system as disclosed herein. The method also comprises exposing the CO₂-derived compound to a gas, such as an inert gas (e.g., H₂). The CO₂-derived compound can be exposed to the gas at a pressure ranging from 200 psi to 800 psi, such as 300 psi to 500 psi, or 350 psi to 450 psi, including 300 psi, 350 psi, 400 psi, 450 psi, 500 psi, 550 psi, 600 psi, 650 psi, 700 psi, 750 psi and 800 psi. In particular disclosed embodiments, the method comprises exposing the CO₂-derived compound to the heterogeneous catalyst system and the gas at a temperature and for a time sufficient to convert the CO₂-derived compound to formate(s), formic acid, or a mixture thereof. For example, in some embodiments, the temperature can range from room temperature (e.g., 19° C. to 25° C.) to 140° C., such as 20° C. to 120° C., 20° C. to 80° C., or 40° C. to 100° C. In exemplary embodiments, the temperature can be 20° C., 40° C., 60° C., 80° C., 100° C., or 120° C. The time of reaction can range from 10 minutes to 15 hours, such as 15 minutes to 10 hours, or 20 minutes to 6 hours, or 30 minutes to 1 hour. In particular disclosed embodiments, a high yield of formate(s), formic acid, or a mixture thereof can be obtained in just under 60 minutes, with particular exemplary embodiments of the method producing high yields under 50 minutes, 40 minutes, or 30 minutes. In some embodiments, the temperature can be increased above room temperature to achieve a shorter reaction time for the conversion of the CO₂-derived compound to formate(s), formic acid, or a mixture thereof. In particular disclosed embodiments, the method does not produce alkyl carbonates as products or intermediates.

The method can further comprise exposing the CO₂-derived compound to a solvent, such as an aqueous solvent, an organic solvent, or a combination thereof. Exemplary solvents that can be used in the methods disclosed herein include water, an alcohol or other organic solvent, or a combination thereof. Suitable alcohols include, but are not limited to ethanol, methanol, 1-propanol, 2-propanol, butanol, isobutanol, pentanol, glycerol, or the like. An exemplary organic solvent is acetone, though other similar organic solvents are contemplated. In some embodiments, a co-solvent system of water and an alcohol, such as ethanol, can be used. In such embodiments, the co-solvent system can comprise 20 wt % to 100 wt % alcohol, such as 20 wt % to 95 wt %, 20 wt % to 90 wt %, 30 wt % to 80 wt % alcohol, or 30 wt % to 70 wt % alcohol. In some embodiments, the amount of alcohol can be 30 wt %, 50 wt %, 60 wt %, 70 wt %, 80 wt %, 90 wt %, 95.6 wt %, or 100%.

In some embodiments, CO₂-derived compounds can be selected from any compound or chemical species derived from CO₂, containing CO₂, or capable of producing CO₂. In some embodiments, the CO₂-derived compound is a compound satisfying a formula (Z)₂CO₃, wherein each Z independently is selected from a metal, hydrogen, ammonium, or a quaternary ammonium group. In some embodiments where the CO₂-derived compound satisfies this formula, the CO₂-derived compound can be selected from a bicarbonate species, such as a metal bicarbonate species (e.g., potassium bicarbonate, sodium bicarbonate, or combinations thereof) or an ammonium bicarbonate; or a carbonate species, such as a metal carbonate species (e.g., potassium carbonate, sodium carbonate, magnesium carbonate, calcium carbonate, or a combination thereof); or an ammonium carbonate. In an independent embodiment, the CO₂-derived compound is not, or is other than, sodium bicarbonate.

In particular disclosed embodiments of the method, the method can further comprise exposing CO₂ to an amine-containing compound to form an amine-captured CO₂-derived compound. Such embodiments of the method can be used to achieve low temperature conversions of amine-captured CO₂ to formate(s). For example, conversion of CO₂ to formate(s) can occur at temperatures as low as 19° C. to 30° C., such as 20° C. to 28° C., or 23° C. to 25° C. The amine-captured CO₂ can be directly hydrogenated using the catalyst systems disclosed herein at these low temperatures.

The amine-containing compound can be any compound capable of reacting with CO₂ to produce a carbamate intermediate. Suitable amine-containing compounds can be selected from ammonia, primary amines, secondary amines, and tertiary amines. In particular disclosed embodiments, the amine-containing compound can be ammonia or a compound having a formula selected from: NH₂R^{a}, wherein R^{a} is aliphatic, such as alkyl, alkenyl, or alkynyl, or heteroaliphatic, such as an aliphatic group substituted with a hydroxyl, a thiol, an alkoxy, a thioether, or an amine group; NH(R^{a})₂, wherein each R^{a} independently is aliphatic, such as alkyl, alkenyl, or alkynyl, heteroaliphatic, such as an aliphatic group substituted with a hydroxyl, a thiol, an alkoxy, a thioether, or an amine group, or wherein both R^{a} groups form, together with the nitrogen atom to which they are attached, a heterocyclic group comprising from 0 to 3 additional heteroatoms (in addition to the nitrogen atom to which each R^{a} is attached); or N(R^{a})₃, wherein each R^{a} independently is aliphatic, such as alkyl, alkenyl, or alkynyl, heteroaliphatic, such as an aliphatic group substituted with a hydroxyl, a thiol, an alkoxy, a thioether, or an amine group, or wherein two or three R^{a} groups form, together with the nitrogen atom to which they are attached, a heterocyclic group comprising from 0 to 3 additional heteroatoms (in addition to the nitrogen atom to which each R^{a} is attached). In particular disclosed embodiments, each R^{a} independently can be selected from C₁₋₁₀alkyl. In some embodiments, one or more R^{a} groups can comprise a terminal hydroxyl group. In additional embodiments, two R^{a} groups can form, together with the amine to which they are attached, a C₃₋₇ heterocycloalkyl group comprising from 1 to 3 heteroatoms in addition to the nitrogen atom to which the R^{a} groups are attached.

In exemplary embodiments, the amine-containing compound can be an alkanolamine selected from monoethanolamine, diethanolamine, triethanolamine, 2-amino-2-methyl-1-propanol, N-methyldiethanolamine, N-methylethanolamine, 1,4-diaminobutane, 1,3-diamino-2-propanol, 2-(diethylamino)ethanol, 1,3-propanediamine, 2-diisopropylamino-ethanol, 2,2-dimethyl-1,3-propanediamine, N-1-methyl-1,3-propanediamine, N-tert-butyldiethanolamine, or combinations thereof. In other embodiments, the amine-containing compound can be a cyclic amine selected from piperazine, piperidine, pyrrolidine, homopiperazine, 1-piperazineethanol, or combinations thereof. In other embodiments, the amine-containing compound can be a mixture of the above-mentioned amines. In an independent embodiment, the amine-containing compound is not, or is other than, N(Hex)₃ or polyethyleneimine.

Particular disclosed embodiments concern using amines having steric bulk to reduce the stability of the carbamate formed between the CO₂ and the amine, thus promoting ready hydrolysis to form a bicarbonate species and ultimately form formate(s), formic acid, or a mixture thereof. In some embodiments, a sterically hindered amine compound can be used to increase the CO₂ absorption capacity of the amine group as such amine compounds form less stable carbamate species as compared to unhindered amine compounds. Increasing the CO₂ absorption capacity of the amine can increase the amount of bicarbonate or carbamate produced, thereby increasing the production of formate(s), formic acid, or a mixture thereof, upon hydrogenation of the CO₂-derived compound.

In yet other embodiments, the method can be used to convert intermediates formed in industrial processes to formate(s), formic acid, or mixtures thereof. For example, certain industrial processes can produce CO₂ as a by-product when producing urea. In such industrial processes, ammonium carbamate is produced as an intermediate that ultimately is converted to urea. The disclosed catalyst systems and methods can be used to convert this ammonium carbamate intermediate to a formate (e.g., ammonium formate), formic acid, or a mixture thereof. In some embodiments, ammonium carbamate can be hydrogenated in the presence of a disclosed catalyst system, H₂, and a solvent. In some embodiments, the solvent can be an aqueous solvent, an organic solvent, or a combination thereof. In particular disclosed embodiments, the solvent can be residual solvent from the initial reaction between ammonia and CO₂, which forms the ammonium carbamate. In particular disclosed embodiments, the reaction is carried out in a single solvent phase rather than a two-phase solvent system.

In embodiments utilizing a carbamate intermediate, the temperature of the reaction can be controlled so as to influence the rate of reaction of the conversion of the carbamate to formic acid, formate(s), or a mixture thereof. In some embodiments, the temperature range at which the conversion of the carbamate to bicarbonate and then formate(s) is carried out can range from 10° C. to 80° C., such as 20° C. to 60° C., or 20° C. to 40° C.

In the method embodiments described above, the amine compounds that are used to capture the CO₂ can be regenerated upon hydrogenation. Thus, the amine compounds can be used to increase the yield of formate(s), formic acid, or mixtures thereof as the regeneration of amines provides the ability to capture more CO₂ in multiple reaction cycles.

In particular disclosed embodiments, the method can further comprise converting formate(s), formic acid, or mixtures thereof to H₂(g). In such embodiments, the method can comprise isolating the formate(s), formic acid, or mixture thereof produced by the conversion of the CO₂-derived compound and further exposing the formate(s), formic acid, or mixture thereof to the catalyst system disclosed herein to dehydrogenate the formate product(s) formed from the CO₂-derived compound. Such a method provides a reversible hydrogen storage and evolution system using the same catalyst system. Formic acid or the derived formate salt(s) produced using the methods disclosed herein can be decomposed via either a dehydrogenation pathway and the hydrogen produced by this pathway can be converted into electrical energy.

### V. Combinations

Also disclosed herein are embodiments of combinations comprising a CO₂-derived compound and a heterogeneous catalyst system comprising Pd and a carbon-based material. In some embodiments, the combinations can further comprise H₂ gas. In additional embodiments, the combinations can further comprise a solvent selected from water, an alcohol, an organic solvent, or a combination thereof. In some embodiments, the combination can comprise a CO₂-derived compound, Pd (e.g., Pd(II) or Pd(0)), activated carbon, formate(s), formic acid, or any mixture thereof.

### VI. Examples

Materials:

The catalyst system samples Pd/AC (5 wt % and 10 wt %), Pd/CaCO₃, Pd/BaSO₄, Pd/Al₂O₃, Ru/AC, Pt/AC, Rh/AC were purchased from Sigma-Aldrich®. Ni/AC was prepared by impregnation method (activated carbon support is VXC-72 purchased from CABOT®). Chemicals such as NH₄HCO₃ (99%), (NH₄)₂CO₃ (99%), NaHCO₃ (99.5%), Na₂CO₃ (>99.5%), KHCO₃ (>99%), K₂CO₃ (>99%), and NH₄CO₂H (>99.5%), used in this paper were also purchased from Sigma-Aldrich®. The chemicals samples ethanolamine (>99%), piperazine (99%), diethanolamine (>98%), triethanolamine (98%), and 2-amino-2-methyl-1-propanol (90%) were also purchased from Sigma-Aldrich®.

### Example I

### Bicarbonate (or Carbonate) Reduction

In this example, low temperature bicarbonate reduction reactions were carried out in a 50 mL stirred Parr micro-reactor. The appropriate amounts of bicarbonate (or carbonate) and catalyst system were added into 20 mL water. The reactor was then sealed, purged with high purity nitrogen three times, and then charged with H₂ to the set pressure. During the reaction, mixing was achieved through an internal propeller operating at 620 RPM. Once the set temperature was attained, the reactor was held at the set temperature for a certain period of time and then quenched in an ice bath to quickly lower the temperature. The reactor was cooled to approximately 20° C., and then the gas pressure was recorded and vented. The reactor was immediately broken down and the liquid was collected for analysis.

The standard reaction conditions were: 20 mL H₂O, 1M concentration of bicarbonate or carbonate, 20° C. reaction temperature, 400 psi (H₂) reaction pressure, 0.1 g catalyst system loading, 1 hour reaction time.

Formate Decomposition—

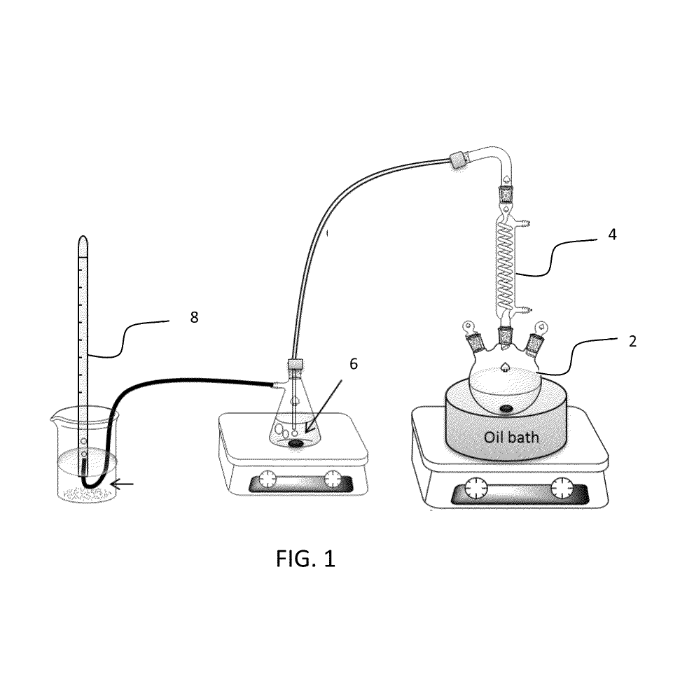

A schematic diagram of the system used in this embodiment is illustrated in FIG. 1. The formate decomposition reaction experiments were carried out in a 50 mL three-necked round bottom flask 2. One neck of the flask was connected to a condenser 4 then further connected to a NaOH solution trap 6 (10 M). Finally, the trap was connected to a gas burette 8. It should be noted that the condenser is used to prevent the volatilization of liquid species, and the NaOH trap is used to adsorb CO₂ generated from the decomposition of bicarbonate. Before reaction, the reaction system was tested to determine its ability to avoid leakage, and then the system was charged with N₂ gas for 5 minutes to make sure no O₂ was in the reaction system.

The released gas during the reaction was passed through the NaOH trap, and its volume was monitored using the gas burette. The catalytic decomposition reaction for the release of hydrogen was initiated by stirring the mixture of the aqueous suspension of catalyst system (0.1 g) in 20 ml NH₄HCO₃ solution (1 M). To ensure accuracy, each reaction was repeated 3 times and the data were averaged.

Catalyst System Stability Testing (for Bicarbonate Reduction)—

The hydrogenation of bicarbonate was carried out in the Parr micro-reactor, the dehydrogenation of formate was carried out in the reactor illustrated in FIG. 1. The fresh catalyst system after one cycle reaction (bicarbonate hydrogenation and formate dehydrogenation) was noted as spent 1 cycle. After every reaction, the spent catalyst system was separated by centrifugation and washed with water and ethanol 5 times, then dried at 50° C. in N₂. The hydrogenation reaction conditions were: 20 mL H₂O, 1M concentration of ammonium bicarbonate, 20° C. reaction temperature, 400 psi (H₂) reaction pressure, 0.1 g fresh and spent catalyst system loading, 1 hour reaction time. Dehydrogenation reaction conditions were: 20 mL H₂O, 1M concentration of ammonium formate, 80° C. reaction temperature, 1 atm (N₂) reaction pressure, 0.1 g catalyst system loading, one hour reaction time.

Aqueous-Phase Product Analysis—

Aqueous samples collected were filtered through a 0.22 μm pore-size filter for high performance liquid chromatography (HPLC) and electrospray ionization mass spectrometer (ESI-MS) analysis. HPLC analysis was performed using a Shimadzu HPLC system equipped with a dual UV-VIS Detector (Shimadzu SPD 10-AV) at 208 and 290 nm and a Refractive Index Detector (Shimadzu RID-10A). For analysis of organic acids and reaction intermediates, the samples were separated in an Aminex 87-H column from Bio-Rad, using 5 mM H₂SO₄ as the mobile phase at 0.7 mL/min flow and a column temperature of 55° C. All samples for ESI-MS analysis were diluted with a base solution containing 0.1 wt % triethylamine and the analysis was performed using a Waters Micromass ZQ quadrupole mass spectrometer. NMR measurements were performed on a 2-channel 400 MHz Varian VNMRS with an ATB automation probe. 1000 scan, decouple, a pulse width of 90° and a recovery delay of 25 seconds. The concentration of different salts in H₂O solution is 1M.

Gas-Phase Product Analysis—

After the reaction, the reactor was cooled until approximately 25° C. The gas pressure was recorded and the gas was collected by a gas bag, and then analyzed by GC. SRI 8610C Gas Chromatograph (Multiple Gas #3 GC) with a TCD detector, oven temperature 200° C. Column 1 was a 3′ Haysep D and Column 2 was a 6′ MS13X.

### Catalyst System Characterization

Transmission Electron Microscope

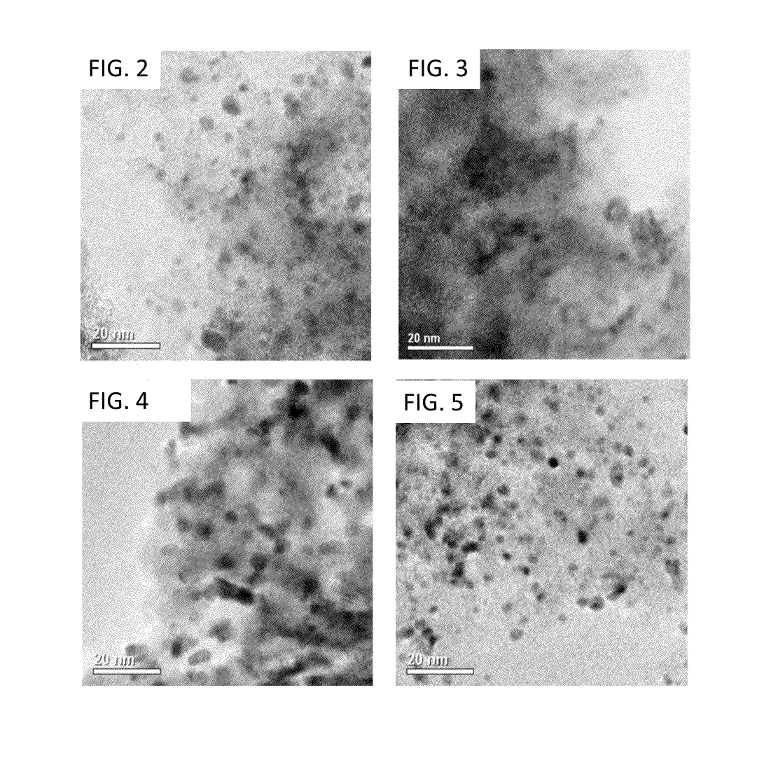

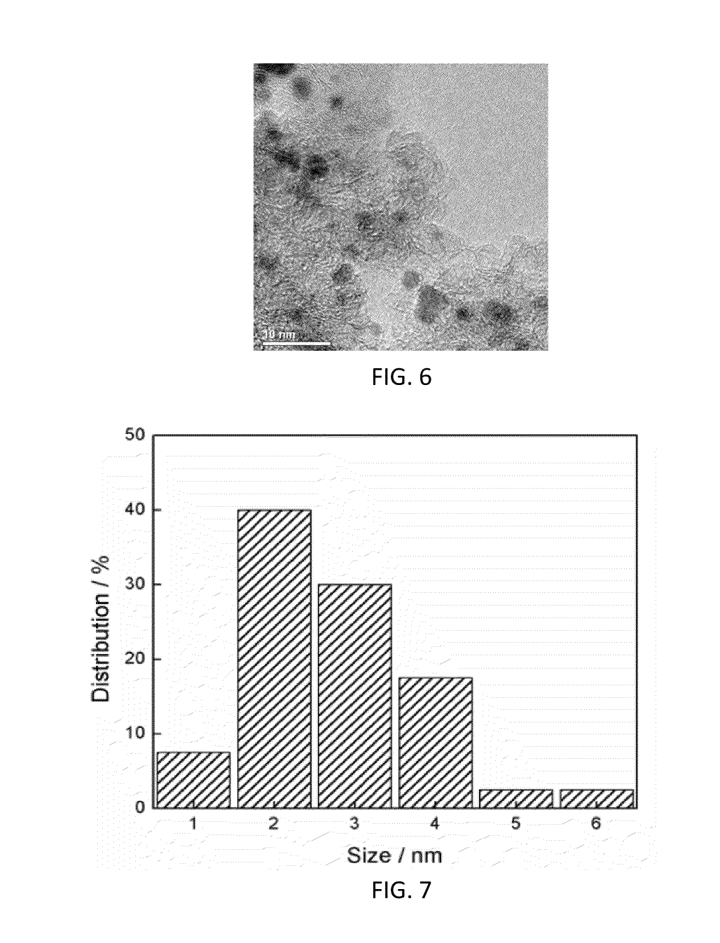

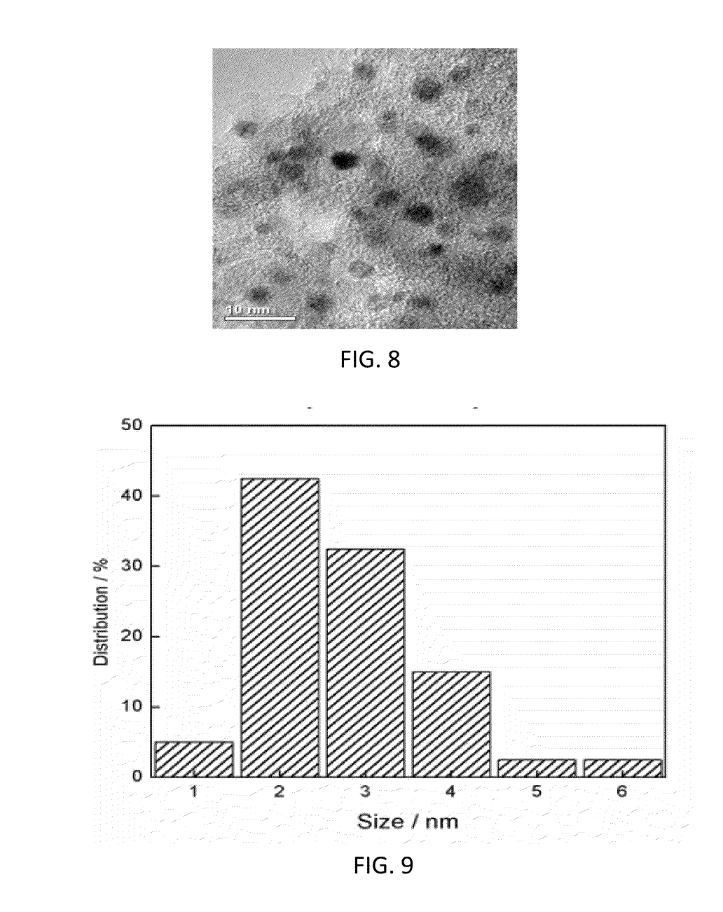

(TEM) was done on Hitachi S-4700 II Scanning Electron Microscope and operated at 200 kv. Results are illustrated in FIGS. 2-5. FIGS. 2-5 are TEM images of the Pd/AC, Pd/Al₂O₃, Ru/AC and Rh/AC, respectively wherein the metal loading is 5 wt % and “AC” stands for activated carbon. SEM images and Pd NPs size distribution graphs of the Pd/AC catalyst system before (fresh catalyst) and after (spent catalyst) 1 cycle of ammonium bicarbonate hydrogenation and then ammonium formate dehydrogenation are provided in FIGS. 6-9. Hydrogenation reaction conditions were as follows: 20° C., 1 hour, 1M bicarbonate in 20 ml H₂O. Dehydrogenation reaction conditions were as follows: 80° C., 1 hour, 1M formate in 20 ml H₂O.

Nuclear Magnetic Resonance (NMR):

NMR measurements were performed on a 2-channel 400 MHz Varian VNMRS with an ATB automation probe. 1000 scan, decouple, a pulse width of 90° and a recovery delay of 25 seconds. The concentration of different salts in H₂O solution was 1M and with 1,4-Dioxane (67.19 ppm) as internal standard.

X-Ray Photoelectron Spectroscopy (XPS):

The XPS measurements were performed on an SSX-100 system (Surface Science Laboratories, Inc.) equipped with a monochromated Al Kα X-ray source, a hemispherical sector analyzer (HSA) and a resistive anode detector. The base pressure was 6.0×10⁻¹⁰ Torr. During the data collection, the pressure was ca. 1.0×10⁻⁸ Torr. Each sample was mounted on a piece of Al sticking tape on a separate sample holder. Care was taken to ensure the surface was fully covered with a sufficiently thick layer of the sample. The X-ray spot size was 1×1 mm², which corresponded to an X-ray power of 200 W. A slight differential charging was found for all samples and a low-energy electron beam (10 eV) was used for charge neutralization. The survey spectra were collected using 10 scans at 150 eV pass energy and 1 eV/step. The high resolution spectra were recorded at 20 scans for the C 1s peak and 80 scans for the Pd 3d peaks using 50 eV pass energy and 0.1 eV/step. For high resolution data, the lowest binding-energy C 1s peak was set at 285.0 eV and used as the reference for the Pd 3d peaks. The curve fitting used a combination of Gaussian/Lorenzian function with the Gaussian percentages being at 80% or higher. Asymmetric peaks were used in fitting the metallic Pd 3d data.

Pulse Chemisorption on Pd/AC Sample—

The analyzer was Micromeritics Autochem II 2920 unit (Table 1). Before the test the sample was pretreated by heating under inert flow Helium (50 mL/min) at 250-350° C. for 60 minutes to remove adsorbed moisture. Then the sample was reduced by 10% H₂ in Ar at 250° C. for 1 hour, followed by helium purge at the same temperature for another 1 hour to remove the physical absorbed H₂ on the surface of the catalyst. CO-pulse chemisorption experiment was carried out at 40° C. using Helium gas with flow rate of 50 mL/min as carrier gas. With recording (0.2 seconds), the defined amount (0.5 mL) of (10% CO in He) was pulsed to the reactor in Helium carrier gas. The above step was repeated until desorption peaks reached the saturation value. The pulsation was terminated when two consecutive CO peaks resulted in an equal amount of CO observed according to the peak area. Between the pulses, the reactor was kept under 50 mL/min Helium flow. (The specific operations were performed following the manual of Micromeritics Autochem II 2920 analyzer).

TABLE 1
Pulse chemisorption analysis results

| Sample (g) | Element | Loading Wt % | Metal Dispersion |
|---|---|---|---|
| Pd/AC | Pd | 5% | 23.3% |
| Pt/AC | Pt | 5% | 42.0% |
| Ru/AC | Ru | 5% | 27.3% |
| Rh/AC | Rh | 5% | 24.3% |
| Ni/AC | Ni | 5% | 16.5% |
| Pd/Al₂O₃ | Pd | 5% | 13.6% |
| Pd/CaCO₃ | Pd | 5% | 10.3% |
| Pd/BaSO₄ | Pd | 5% | 5.8% |

In one example, the hydrogenation of bicarbonate and carbonate salts with different cations, Na⁺, K⁺, and NH₄⁺ was determined. As shown in Table 2, formates can be easily produced from the hydrogenation of the bicarbonates with all three different cations, but cannot be yielded from carbonates, except from (NH₄)₂CO₃, over the Pd on carbon nano-catalyst system at room temperature. It was more difficult to hydrogenate carbonate salts than bicarbonate salts since the protonation of carbonate ions was considered as the rate limiting step in aqueous solutions, especially at low temperatures.

In the hydrogenation reaction system using a Pd/AC (5 wt % Pd) nano-catalyst system and 20 mmol NH₄HCO₃ in 20 ml H₂O (NH₄HCO₃ concentration=1 mol/L), the ability to produce formate was determined. A high yield of ammonium formate, ˜59.6%, with a TON of 1103 was gained after reacting for 1 hour when the initial H₂ pressure was 5.5 MPa. By extending the reaction time to 2 hours, a 90.4% formate yield with a TON of 1672 was obtained. As a side-by-side comparison, a [{RuCl₂(benzene)}₂] homogeneous catalyst was used under the similar reaction conditions (5 MPa initial H₂ pressure and a 2-hour reaction time). Using the [{RuCl₂(benzene)}₂] homogeneous catalyst a yield of formate 35% with a TON of 807 was gained from 24 mmol NaHCO₃ in the solution of 25 ml H₂O and 5 ml THF solvent (reagent concentration<1 mol/L). These results indicate that the hydrogen storage process based on the reduction of ammonium bicarbonate over the Pd/AC heterogeneous catalyst system is more efficient than a homogeneous catalyst.

TABLE 2
Catalytic hydrogenation of bicarbonates and carbonates

| | | | Reaction | | | |
|---|---|---|---|---|---|---|
| | | | conditions | | | |
| | | | Pressure | | | |
| | | | (H₂) | Time | Yield | |
| Entry | Regent | Catalyst^{[b]} | (MPa) | (h) | (%) | TON^{[c]} |
| 1 | NaHCO₃ | Pd/AC | 2.75 | 1 | 28.6 | 527 |
| 2 | Na₂CO₃ | Pd/AC | 2.75 | 1 | 0.05 | <1 |
| 3 | KHCO₃ | Pd/AC | 2.75 | 1 | 30.8 | 567 |
| 4 | K₂CO₃ | Pd/AC | 2.75 | 1 | 0.07 | <1 |
| 5 | NH₄HCO₃ | Pd/AC | 2.75 | 1 | 42.4 | 782 |
| 6 | (NH₄)₂CO₃ | Pd/AC | 2.75 | 1 | 15.1 | 278 |
| 7 | NH₄HCO₃ | Pd/AC | 2.75 | 6 | 84.9 | 1571 |
| 8 | NH₄HCO₃ | Pd/AC | 2.75 | 15 | 95.6 | 1769 |
| 9 | NH₄HCO₃ | Pd/AC | 0.69 | 1 | 16.9 | 312 |
| 10 | NH₄HCO₃ | Pd/AC | 1.38 | 1 | 31.3 | 579 |
| 11 | NH₄HCO₃ | Pd/AC | 4.14 | 1 | 53.1 | 982 |
| 12 | NH₄HCO₃ | Pd/AC | 5.52 | 1 | 59.6 | 1103 |
| 13 | NH₄HCO₃ | Pd/AC | 5.52 | 2 | 90.4 | 1672 |
| 14 | NH₄HCO₃ | Ru/AC | 2.75 | 1 | 0.2 | 3 |
| 15 | NH₄HCO₃ | Rh/AC | 2.75 | 1 | 0.2 | 3 |
| 16 | NH₄HCO₃ | Pt/AC | 2.75 | 1 | 0 | 0 |
| 17 | NH₄HCO₃ | Ni/AC | 2.75 | 1 | 0 | 0 |
| 18 | NH₄HCO₃ | Pd/Al₂O₃ | 2.75 | 1 | 8.9 | 278 |
| 19 | NH₄HCO₃ | Pd/CaCO₃ | 2.75 | 1 | 0.6 | 20 |
| 20 | NH₄HCO₃ | Pd/BaSO₄ | 2.75 | 1 | 2.9 | 212 |

In other examples, the hydrogenation of ammonium bicarbonate with different supported metal catalysts was explored. Carbon material supported transition metal catalysts, such as Pd/AC, Ru/AC, Rh/AC, Pt/AC and Ni/AC, were used. In the hydrogenation of ammonium bicarbonate, it was determined that only Pd catalysts showed catalytic activity, while other transition metals such as Ru, Rh, Pt and Ni were inactive under the hydrogenation conditions utilized in this example (Table 2 entries 8 and 14-17). The active carbon support was superior to other types of support materials, including Al₂O₃, CaCO₃ and BaSO₄ (Table 2, entries 18-20).

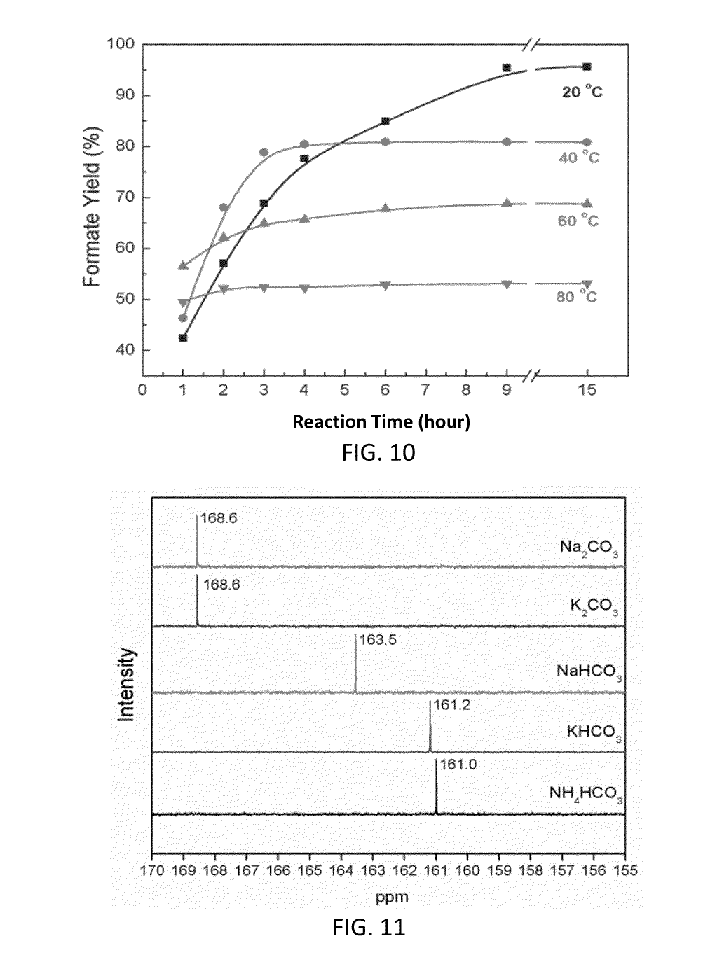

In another example, the catalyst system materials (metals and supports), process conditions were manipulated. In reactions at a higher H₂ gas pressure of 2.75 MPa, the equilibrium ratio of NH₄HCO₂ to NH₄HCO₃ was shifted significantly to approximately 95:5 in 15 hours (Table 2, entry 8). On the other hand, with increasing the reaction temperature from 20° C. to 80° C., the hydrogenation rates increased but the equilibrium yield of formate decreased from ˜95% to ˜50%, as shown in FIG. 10. Higher reaction temperatures favor the dehydrogenation reaction and shift the equilibrium to hydrogen evolution. A high formate yield was achieved at room temperature, implying that the disclosed reaction system does not need additional energy consumption for heating.

To verify the current theory that the cation effect may influence the amount of formate produced, ¹³C NMR spectra were recorded (FIG. 11). The peaks from 155 ppm to 170 ppm in ¹³C NMR spectra were assigned to the bicarbonate/carbonate ion pair with fast proton exchange. The ratio of HCO₃⁻ to CO₃²⁻ has an effect on the chemical shift. As the ratio increases, the peak of the bicarbonate/carbonate ion pair shifts to low ppm positions. As shown in FIG. 11, bicarbonate salts, such as NaHCO₃, KHCO₃ and NH₄HCO₃, have a higher HCO₃⁻ concentration than carbonate salts (Na₂CO₃ and K₂CO₃), and the peaks of the ion pair are located at low ppm positions for bicarbonate salts. The peak positions of the bicarbonate salts suggest that the concentrations of bicarbonate ions are in the order of: NaHCO₃ (0.61M)<KHCO₃ (0.89 M)<NH₄HCO₃ (0.92 M) (Table 3). Thus NH₄HCO₃ has a high concentration of HCO₃⁻ in the aqueous solution, which led to a high formate yield of hydrogenation reaction.

TABLE 3
The concentration of bicarbonate ion in different bicarbonate

| | HCO₃⁻concentration | CO₃²-concentration |
|---|---|---|
| Bicarbonate | (M) | (M) |
| NaHCO₃ | 0.61 | 0.39 |
| KHCO₃ | 0.89 | 0.11 |
| NH₄HCO₃ | 0.92 | 0.08 |

The concentrations were calculated by the modified equation:
$[ \frac{{HCO}_{3}^{-}}{{CO}_{3}^{2 -}} ] = \frac{168.6 - S}{S - 160.3}$ .
The S in this equation is the ¹³C-NMR position of different bicarbonate samples.

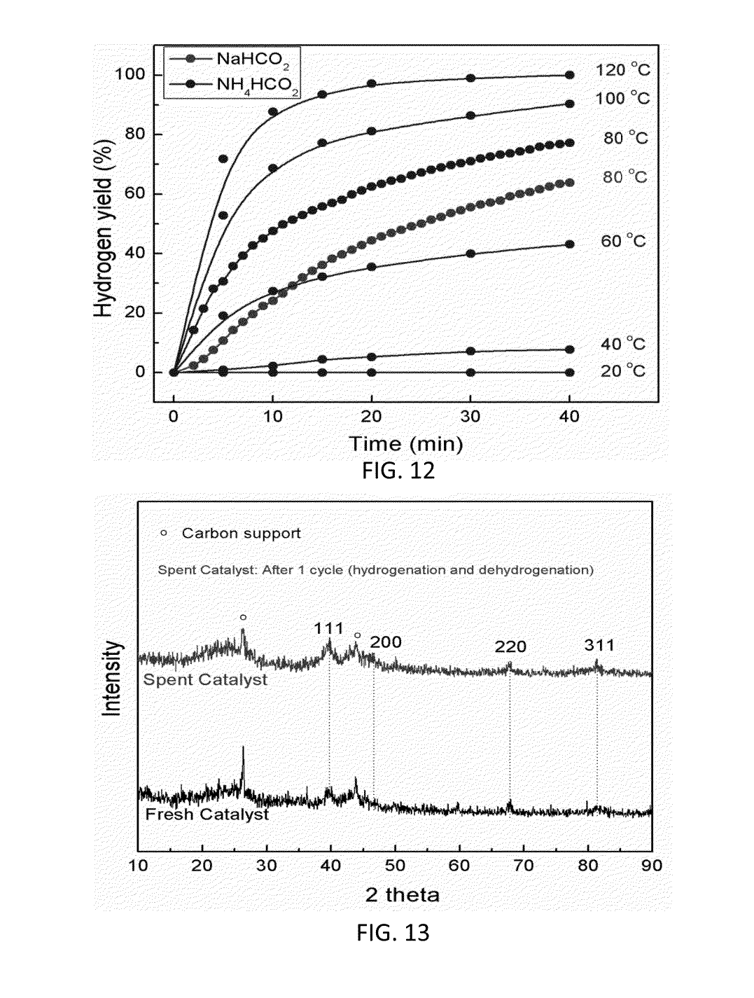

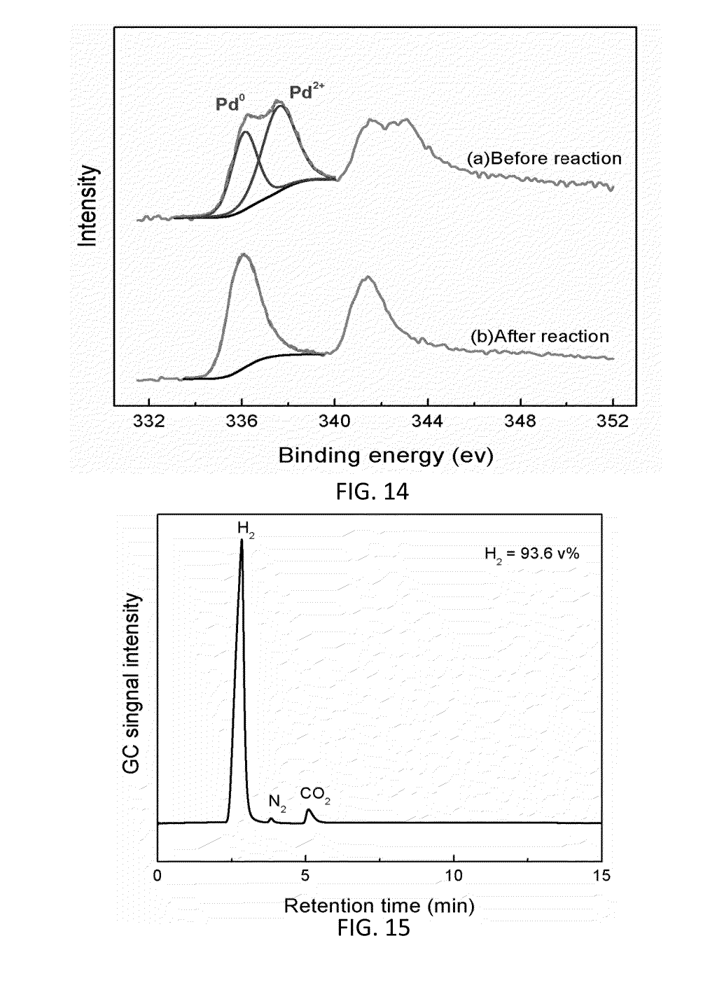

The dehydrogenation of ammonium formate, which closes the hydrogen storage/evolution cycle, also was explored. Reaction temperature and H₂ pressure are also factors that can control the reaction equilibrium. Low reaction temperature and high H₂ pressure favor the NH₄HCO₃ hydrogenation. Contrarily, high temperature and low H₂ pressure should favor the dehydrogenation of NH₄HCO₂. Accordingly, the dehydrogenation of NH₄HCO₂ at a high temperature range in an N₂ atmosphere with an initial pressure of 1 atm was performed. As shown in FIG. 12, no decomposition reaction occurred at 20° C. As the reaction temperature increased to 80° C., the yield of hydrogen reached 63% and 77% after 20 and 40 minutes, respectively, and even reached 92.1% after 1.5 hours reaction (Table 4). The comparison of the hydrogen evolution efficiencies of NH₄HCO₂ and NaHCO₂, as shown in FIG. 12, illustrates that the yield of hydrogen from NaHCO₂ only reached 44% after 20 minutes at 80° C. Thus the hydrogen evolution efficiency of NaHCO₂ is only 70% of that of NH₄HCO₂ at the beginning stage of reaction (0-20 min). The NH₄⁺ ion effect on the dehydrogenation reaction may attribute to the higher hydrogen evolution efficiency of NH₄HCO₂ than that of NaHCO₂. At elevated temperatures, NH₄HCO₂ can be decomposed to NH₃, H₂O, and CO₂ and then rehydrated to form NH₄HCO₃. As the reaction temperature increased further, the generation of H₂ gas from NH₄HCO₂ became much faster. For example, when the reaction temperature reached 120° C., the yield of H₂ reached 97% within 20 minutes, and after 40 minutes the yield of H₂ was almost 100%. Similar to the hydrogenation of bicarbonate, Pd/AC is also the best among the tested catalysts for formate dehydrogenation (Table 4), which makes it highly feasible to build the reversible hydrogen storage-evolution system using the same catalyst. The TEM images and XRD patterns indicated that the Pd/AC catalysts were very stable. As shown in FIGS. 6-9, after one cycle of the complete reaction (hydrogenation of ammonium bicarbonate and then dehydrogenation of ammonium formate without the regeneration of the spent catalyst), no obvious sintering or aggregation of the Pd NPs was observed. The XRD patterns of Pd/AC catalysts (shown in FIG. 13) reveal that the peaks, which are assigned to the lattice planes [111], [220], [311] and [200] of Pd, are almost unchanged before and after one cycle reaction. At the 5^{th} cycle, the catalyst system activity was as high as that using the fresh catalyst, as shown in Table 5. The XPS spectra of the fresh and spent Pd/AC samples show that the Pd²⁺ in the fresh catalyst system was completely reduced to Pd⁰ after one cycle of reaction, as shown in FIG. 14.

TABLE 4
The results of ammonium formate decomposition with different
catalyst

| | | | Reaction conditions | | | |
|---|---|---|---|---|---|---|
| | | | Initial | | | |
| | Catalyst^{a} | | Pressure | Time | Temp. | H₂ yield |
| Entry | (g) | | (atm N₂) | (h) | (° C.) | (%) |
| 1 | — | | 1 | 1 | 80 | 0.06 |
| 2 | Pt/AC | 0.1 | 1 | 1 | 80 | 3.12 |
| 3 | Ru/AC | 0.1 | 1 | 1 | 80 | 0.32 |
| 4 | Rh/AC | 0.1 | 1 | 1 | 80 | 0.45 |
| 5 | Pd/Al₂O₃ | 0.1 | 1 | 1 | 80 | 2.96 |
| 6 | Pd/AC | 0.1 | 1 | 1 | 80 | 85.6 |
| 7 | Pd/AC | 0.1 | 1 | 1.5 | 80 | 92.1 |

TABLE 5
The stability testing of Pd/AC catalyst system

Results

| | | Hydrogenation | Dehydrogenation |
|---|---|---|---|
| | | reaction Formate | reaction H₂ |
| Entry | Catalyst | Yield (%) | yield (%) |
| 1 | Fresh Pd/AC | 42.4 | 85.6 |
| 2 | Spent 1 | 42.6 | 86.1 |
| 3 | Spent 2 | 42.0 | 84.2 |
| 4 | Spent 3 | 40.5 | 84.9 |
| 5 | Spent 4 | 40.6 | 84.6 |

FIG. 15 indicates that H₂, N₂ and CO₂ were gaseous products and the volume percentage of H₂ was 93.6%. No CO was detected, implying that the Pd/AC catalyst system selectively catalysed the dehydrogenation reaction. The very low amount of CO₂ and a trace amount of NH₃ likely result as by-products from the decomposition of NH₄HCO₂ and NH₄HCO₃ at elevated reaction temperatures. Any residual NH₃ can be removed easily by passing the gas stream through a scrubber containing acid solutions. The examples above demonstrate that the ammonium bicarbonate/formate redox equilibrium system is a feasible system for reversible hydrogen storage and evolution. High yields of formates and hydrogen were obtained from the hydrogenation of NH₄HCO₃ and the dehydrogenation of NH₄HCO₂, respectively, using the same Pd/AC nano-catalyst. Reaction temperature and H₂ pressure are key factors in controlling the hydrogen storage/evolution equilibrium in this system. Up to 96% NH₄HCO₂ yield was achieved when the hydrogenation reaction was carried out at room temperature and an initial H₂ pressure of 2.75 MPa, while nearly 100% H₂ yield was obtained from the dehydrogenation of NH₄HCO₂ at 80° C. and initial N₂ pressure of 0.1 MPa. Compared to the homogeneous catalytic system, the disclosed heterogeneous catalyst systems have the following advantages: no organic solvents or inorganic additives are needed; high energy density hydrogen (stored in ammonium formate salts) are easily transported and distributed; and the Pd/AC catalyst system is very stable, being more easily recycled and handled than homogeneous catalysts.

### Example 2

CO₂ Capture with Amine:

CO₂ capture was carried out in a 50 mL flask with a magnetic stifling system at 500 RPM. 20 mL amine solution (1M), were charged into the flask and control the temperature of absorbent solution at 20° C. with a water bath. Then, bubbling with 150 ml/min CO₂ gas into the amine solution. The amount of CO₂ absorbed was determined by an analytical valance every five minutes.

Hydrogenation of Amine-Captured CO₂:

The low temperature hydrogenation of amine-captured CO₂ experiments were carried out in the 50 mL stirred Parr micro-reactor. The appropriate amounts of CO₂ amine solution and catalyst system were added into reactor. The reactor was then sealed, purged with high purity nitrogen three times, and then charged with the H₂ to the set pressure. During the reaction, mixing was achieved through an internal propeller operating at 620 RPM, or 1520 RPM in certain embodiments. Once the set temperature was attained, the reactor was held at the set temperature for a certain period of time and then quenched in an ice bath to quickly lower the temperature. The reactor was cooled until approximately 20° C., and then the gas pressure was recorded and vented. The reactor was immediately broken down and the liquid was collected for analysis. The standard reaction conditions were: CO₂ amine solution (20 ml), the concentrations of bicarbonate or carbonate were 1M in some embodiments, reaction temperature was 20° C., reaction pressure was 400 psi (H₂), and catalyst system loading was 0.1 g, reaction time is 1 hour.

The formate yield was calculated on the carbon basis and defined as follows:

$$Yield of formate [ \% - C ] = \frac{{mol}_{product} \times C atoms in product}{{mol}_{reactant charged} \times C atoms in reactant} \times 100$$

Product Analysis:

Aqueous samples collected were filtered through a 0.22 μm pore-size filter for high performance liquid chromatography (HPLC) and electrospray ionization mass spectrometer (ESI-MS) analysis. HPLC analysis was performed using a Shimadzu HPLC system equipped with a dual UV-VIS Detector (Shimadzu SPD 10-AV) at 208 and 290 nm and a Refractive Index Detector (Shimadzu RID-10A). For analysis of organic acids and reaction intermediates, the samples were separated in an Aminex 87-H column from Bio-Rad, using 5 mM H₂SO₄ as the mobile phase at 0.7 mL/min flow and a column temperature of 55° C. All samples for ESI-MS analysis were diluted with a base solution containing 0.1 wt % triethylamine and the analysis was performed using a Waters Micromass ZQ quadrupole mass spectrometer.

Catalyst System Characterization:

Transmission Electron Microscope (TEM) was done on Hitachi S-4700 II Scanning Electron Microscope operated at 200 kV. The crystalline structure and the size of nano-catalysts was characterized by a PANalytical X'Pert PRO diffractometer (Cu Kα radiation, λ=0.15418 nm) at 45 kV and 40 mA. The XPS measurements were performed on an SSX-100 system (Surface Science Laboratories, Inc.) equipped with a monochromated Al Kα X-ray source, a hemispherical sector analyser (HSA) and a resistive anode detector. The base pressure was 6.0×10-10 Torr. During the data collection, the pressure was ca. 1.0×10-8 Torr. Each sample was mounted on a piece of Al sticking tape on a separate sample holder. The X-ray spot size was 1×1 mm², which corresponded to an X-ray power of 200 W. A slight differential charging was found for all samples and a low-energy electron beam (10 eV) was used for charge neutralization. The survey spectra were collected using 10 scans at 150 eV pass energy and 1 eV/step. The high resolution spectra were recorded at 20 scans for the C 1s peak and 80 scans for the Pd 3d peaks using 50 eV pass energy and 0.1 eV/step. For high resolution data, the lowest binding-energy C is peak was set at 285.0 eV and used as the reference for the Pd 3d peaks. The curve fitting used a combination of Gaussian/Lorenzian function with the Gaussian percentages being at 80% or higher. Asymmetric factors were used in fitting some of the Pd 3d data.

To establish that methods disclosed herein can be combined with current CO₂ capture and storage process and use amine-captured CO₂ as reactant directly, a solution of AMP was used to capture CO₂ gas first, and then the captured CO₂ was hydrogenated with H₂.

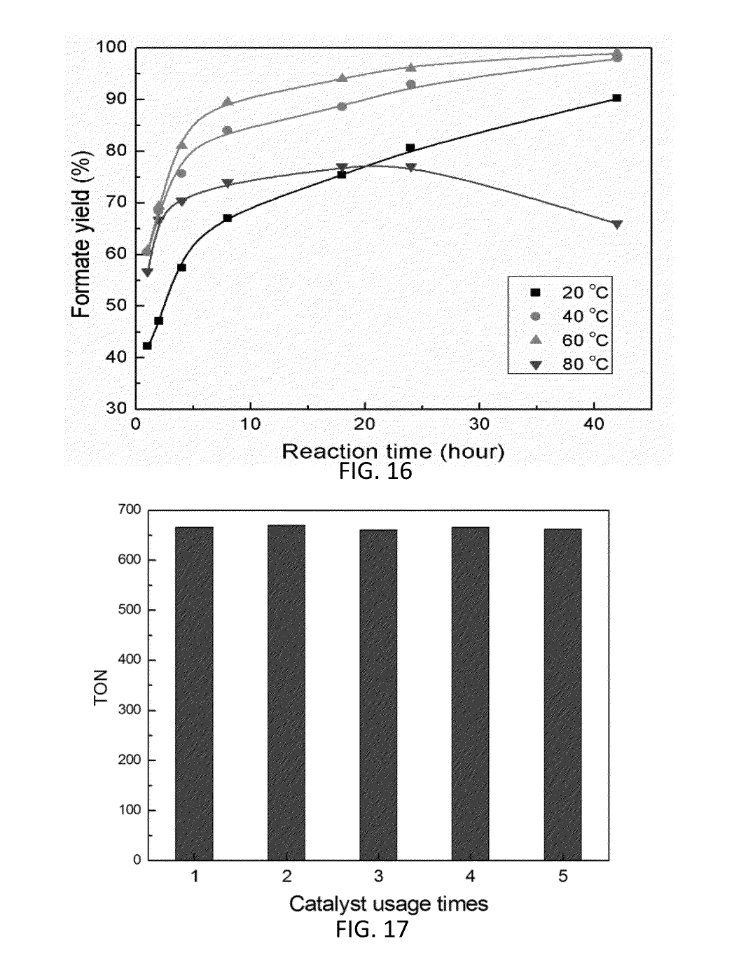

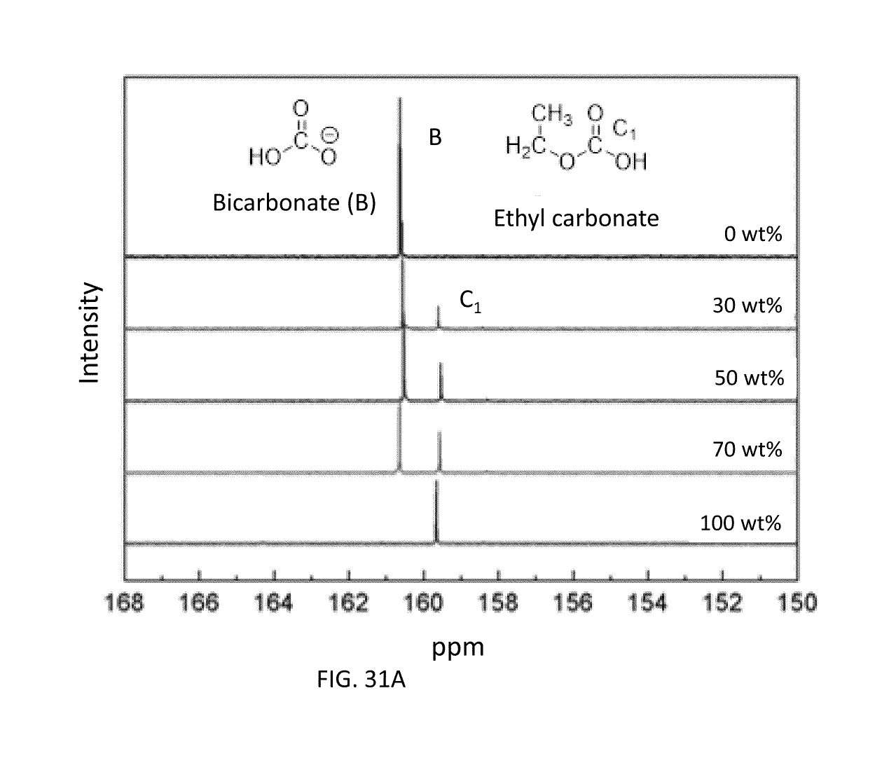

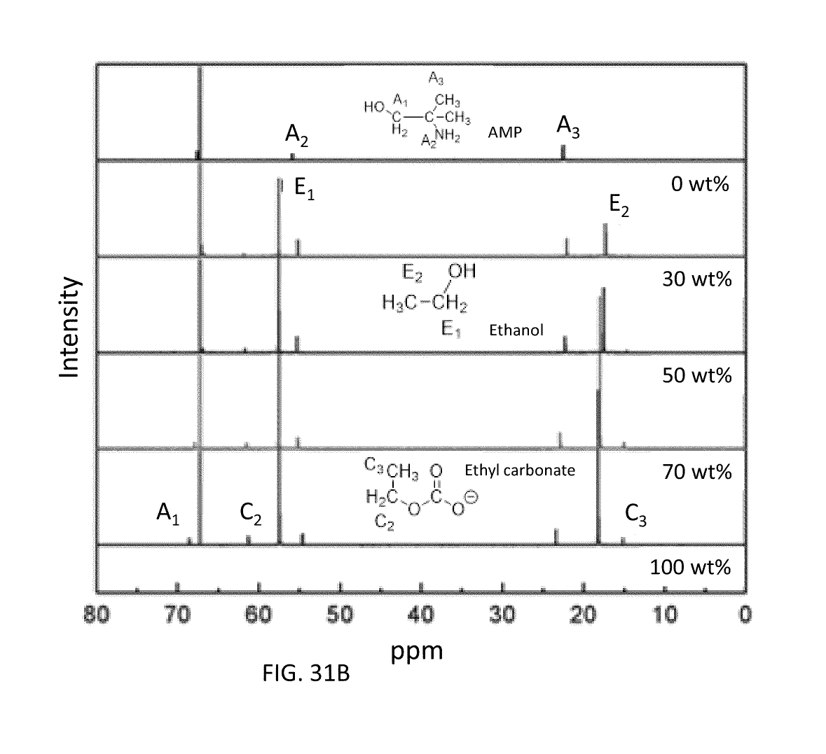

Tables 6 and 7, FIG. 16 and FIG. 17 show the results of catalytic hydrogenation of AMP-captured CO₂ in alcohol-water solutions (wherein ethanol was present in 70 wt %). FIGS. 31A and 31B are ¹³C-NMR spectra illustrating the progress of the different reactions using the different alcohol-water solutions.

In this example, the transition metals Ru, Rh, Pt and Ni were inactive under the test hydrogenation conditions. Pd-containing embodiments exhibited a high activity. The active carbon support also was superior to other supports including Al₂O₃, and CaCO₃. In some embodiments, the rate of hydrogenation was maintained at, or substantially similar to, the rate of the CO₂ capture rate by adjusting the reaction conditions, such as by adjusting the catalyst, the H₂ pressure, and reaction temperature.

TABLE 6
Catalytic hydrogenation of AMP captured CO2^{[a]}

| | Reaction conditions ^{[b]} | | | | | | |
|---|---|---|---|---|---|---|---|
| | Solvent^{[c]} | | | | | Formate | |
| | (wt % | | T | P H₂ | Time | Yield | |
| Entry | EtOH) | Catalyst | (° C.) | (psi) | (h) | (%) | TON^{[d]} |
| 1 | 70 | Pd/AC | 20 | 400 | 1 | 42.2 | 666 |
| 2 | 70 | Ni/AC | 20 | 400 | 1 | 0 | 0 |
| 3 | 70 | Pt/AC | 20 | 400 | 1 | 0 | 0 |
| 4 | 70 | Ru/AC | 20 | 400 | 1 | 0 | 0 |
| 5 | 70 | Rh/AC | 20 | 400 | 1 | 0 | 0 |
| 6 | 70 | Pd/Al₂O₃ | 20 | 400 | 1 | 8.6 | 136 |
| 7 | 70 | Pd/CaCO₃ | 20 | 400 | 1 | 0 | 0 |
| 8 | 70 | Pd/AC | 20 | 400 | 2 | 47.1 | 743 |
| 9 | 70 | Pd/AC | 20 | 400 | 4 | 57.4 | 906 |
| 10 | 70 | Pd/AC | 20 | 400 | 24 | 80.6 | 1272 |
| 11 | 70 | Pd/AC | 20 | 400 | 42 | 90.3 | 1425 |
| 12 | 70 | Pd/AC | 40 | 400 | 24 | 93.0 | 1468 |
| 13 | 70 | Pd/AC | 60 | 400 | 24 | 95.7 | 1511 |
| 14 | 70 | Pd/AC | 80 | 400 | 24 | 77.0 | 1215 |
| 15 | 70 | Pd/AC | 20 | 200 | 1 | 20.0 | 316 |
| 16 | 70 | Pd/AC | 20 | 300 | 1 | 29.0 | 458 |
| 17 | 70 | Pd/AC | 20 | 600 | 1 | 44.5 | 702 |
| 18 | 70 | Pd/AC | 20 | 800 | 1 | 45.0 | 710 |

^{[a]}AMP concentration is 1M, and CO₂ capture capacity (mole CO₂/mole amine) is 0.96.
^{[b]}Reaction conditions: 20 ml capture solution, 0.1 g catalyst system (the metal loading of all the catalyst system is 5 wt %).
^{[c]}Solvent (for both capture and hydrogenation) is ethanol-water co-solvent, 70 wt % means the ratio of anhydrous ethanol and water is 70/30.
^{[d]}TON was calculated by the formula: total mole number of formate formed/(total number of Pd atom × surface dispersion)) Surface dispersion was calculated by the formula: surface Pd atoms/total Pd atoms, this result is come from the carbon monoxide chemisorption analysis (Table 1).

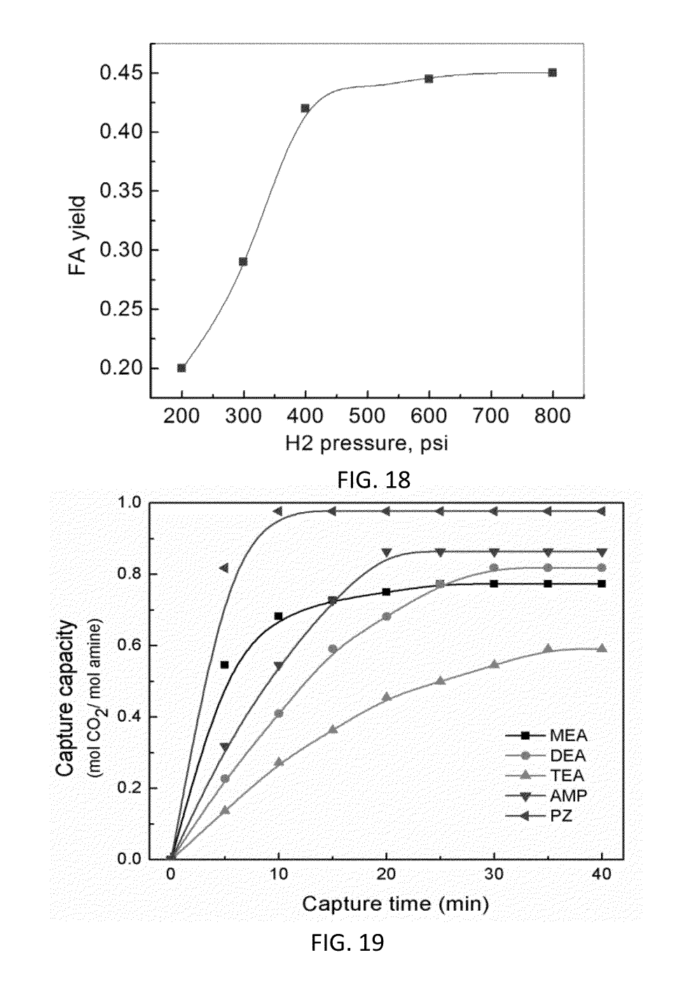

Besides catalyst system type (metal and support), initial H₂ pressure, reaction temperature and reaction time are also are factors that can affect the hydrogenation of AMP captured CO₂. H₂ initial pressure may have a great effect on the H₂ solubility in co-solvent. For example, it was determined that increased H₂ pressure (such as from 200 psi to 400 psi), improved the formate yield from 20.0% to 42.2% for 1 hour reaction (Table 6, entries 1 and 15, and FIG. 18) and from 39.0% to 82.5% (Table 7, entries 7 and 10). Further increases in H₂ pressure (such as from 400 psi to 800 psi, such as 600 psi) only increased the formate yield to 45% (Table 6, entry 18) and 91.8% (Table 7, entry 11), which may be due to the fact that the reaction efficiency was restricted by the active Pd sites amount. In some conventional methods, a 1:1 hydrogenation reaction equilibrium ratio of formate/bicarbonate was obtained when purging the NaHCO₃ solution with 0.1 MPa H₂ gas for a long time run (40-90 hours). However, in this example, at a higher H₂ gas pressure of 400 psi, the formate yield was shifted significantly to approximately 90% in 42 hours (Table 6, entry 11) and 100% in 2.6 hours (Table 7, entry 9). The equilibrium yield could shift to nearly >99:1 in some embodiments. By increasing the reaction temperature from 20° C. to 60° C., the hydrogenation rates increased and equilibrium yield of formate could reach 99% at 42 reaction hours, as shown in FIG. 16. Further increasing the reaction temperature to 80° C. can shift the equilibrium yield and decrease the amount of formate to around 70% (FIG. 16). Higher reaction temperatures favor the dehydrogenation reaction and shift the equilibrium to hydrogen evolution. In one example, a high formate yield was achieved at a low temperature of 20-60° C. In one example, increasing the reaction temperature to 40° C. resulted in an equilibrium yield decrease to around 68.5%. Accordingly, the methods and catalyst systems disclosed herein can be used to directly convert an amine-captured CO₂ solution to formate; such amine-captured CO₂ solutions typically are obtained from industrial processes at 60° C. Therefore, additional energy consumption for heating is not needed in the disclosed methods.

In one embodiment, the disclosed heterogeneous catalyst system and method was compared with that of a homogeneous catalyst to hydrogenate PEI-captured CO₂. In contrast to the homogeneous catalyst, which produced a TON of 726 and formate yield of 55% (at 4 MPa, 580.15 psi, H₂, and at 60° C. for 16 hours), a heterogeneous catalyst system as disclosed above produced a 89.5% formate yield and a TON of 1412 was obtained in only 8 hours at 60° C. reaction (FIG. 16). These results indicate that the hydrogenation amine-captured CO₂ over the Pd/AC heterogeneous catalyst system can be more efficient than the homogeneous catalyst conventionally used.

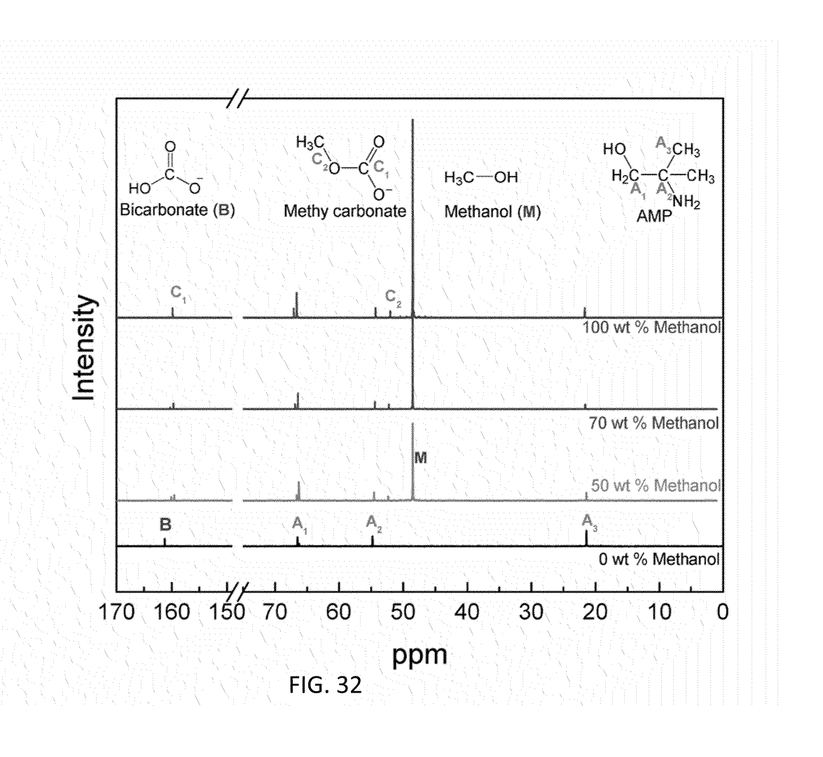

Another advantage of the heterogeneous catalysts disclosed herein is the stability of the catalyst system and amine CO₂ capture agent. As shown in FIG. 17, after 5 repeated reactions without regeneration, the catalyst system had no activity loss compared to the fresh catalyst, indicating that the Pd/AC catalyst system is very stable. Additionally, the amine CO₂ capture agent was resistant to degradation. The degradation of the amine is one disadvantage for CO₂ capture and storage process. Two main types of degradation have been studied: thermal degradation, which occurs at high temperature and high CO₂ partial pressure; and oxidative degradation. Oxidative degradation is mainly due to the presence of a large amount of 02 in flue gases. However, in this example and as disclosed herein, the present methods of CO₂ hydrogenation using the heterogeneous catalysts was carried out at low temperature (20-60° C.) and in presence of H₂, so neither thermal degradation nor oxidative degradation occur (FIG. 32).

TABLE 7
The result of hydrogenation of captured CO₂ by AMP in different solvents.

| | Capture ^{[a]} and | Captured CO₂ species concentration (M)^{[b]} | | | | | |
|---|---|---|---|---|---|---|---|
| | Hydrogenation^{[b]} | | | | Ethyl-CO₂⁻ | Conversion results | |
| | Solvent | HCO₃⁻ | CO₃²⁻ | RNCO²⁻ | Ethyl | Formate Yield | |
| Entry | (wt % ETOH) | Bicarbonate | Carbonate | Carbamate | Carbonate | (%) | TON^{[d]} |
| 1 | 0 | 0.93 | 0.03 | 0.00 | 0 | 19.8 | 303 |
| 2 | 30 | 0.90 | 0.01 | 0.01 | 0.04 | 23.1 | 365 |
| 3 | 50 | 0.73 | 0.01 | 0.01 | 0.21 | 34.1 | 538 |
| 4 | 60 | 0.45 | 0.00 | 0.03 | 0.48 | 35.7 | 564 |
| 5 | 70 | 0.32 | 0.00 | 0.03 | 0.61 | 42.5 | 650 |
| 6 | 80 | 0.18 | 0.00 | 0.03 | 0.75 | 47.8 | 722 |
| 7 | 90 | 0.03 | 0.00 | 0.03 | 0.90 | 49.4 | 753 |
| 8 | 95.6 | 0.01 | 0.00 | 0.03 | 0.92 | 50.5 | 777 |
| 9 | 100 | 0.00 | 0.00 | 0.03 | 0.93 | 38.1 | 586 |

^{[a]} CO₂ capture conditions: 20 ml amine/water-ethanol solution, concentration of AMP is 1M, wt % ETOH of the capture-solvent is the same as hydrogenation-solvent, capture temperature is 20° C., capture time is 40 min.
^{[b]}Hydrogenation conditions: after capture CO₂, directly transmit the 20 ml amine/water solution in to Parr reactor. Catalyst Pd Nps on carbon (5 wt %) is 0.1 g, reaction temperature is 20° C., Hydrogen pressure is 400 psi, reaction time is 1 hour.
^{[c]}The concentration of different captured CO₂ species was got from NMR spectra.
^{[d]}TON was calculated by the formula: [total mole number of formate formed/(total mole number of Pd atom × 23.2%)] the 23.2% is the dispersion of Pd atom on the surface of Pd NPs, this result is come from the carbon monoxide chemisorption.

Table 7 (above) shows the results of catalytic hydrogenation of AMP captured CO₂ in different ethanol-water solutions. The yield of formate was 19.8% and the TON reached 303 with the activated carbon supported palladium catalyst system (5% Pd/AC) after reacting for 1 hour in water at 20° C. (Table 7, entry 1). Adding alcohol in the water solvent improved the hydrogenation performance of AMP captured CO₂ and in some embodiments, the azeotrope ethanol (95.6 wt % ethanol) provided a 50.5% formate yield, with a TON reaching 777. Since the hydrogenation of AMP captured CO₂ is a gas/liquid/solid multiphasic reaction system, the diffusion of the gas reactant, H₂, could be the rate-limiting step due to its low solubility in liquid phase. However, including ethanol as a co-solvent (at least in part) improves the yield as the solubility of hydrogen in ethanol is one magnitude larger than it in water. So increasing the proportion of ethanol can facilitate the hydrogeantion reaction. It is also currently believed that the co-solvent can effect the distribution of the active intermediate of the amine-captured CO₂ species for the hydrogenation reaction. As shown in FIG. 31A, there is only peak located at 161.2 ppm, which belongs to bicarbonate/carbonate ion pair in water. The introduction of ethanol as co-solvent creates a new group of ¹³C NMR peaks located at 160.5 to 159.5 ppm, which indicates formation of a new carbon species. The formate yield in pure ethanol was 38.1%, which may indicate that this new species also could be hydrogenated. The solubility of AMP formate decreased in pure ethanol, which can explain the yield decrease in formate (decease from 50.5% in 95.6 wt % ethanol to 38.1% in pure ethanol). In some embodiments, mass transfer also had an effect on the yield of the hydrogenation reaction. High speed stir rate on different solvents was examined (FIG. 32). Above a stirring rate of 700 rpm in 70% ETOH, the reaction rate is practically independent of the speed of agitation, and it can be assumed that in this region the reaction is chemically controlled. However, in some embodiments, 700 rpm was not enough for the solvent of 80%-100% ETOH system. Also, the good solubility of amines and low viscosity are likely advantages of a solvent for both capture and hydrogenation process. Low viscosity also improves the mass transfer, therefore increasing the absorption and hydrogenation reaction efficiency. It is currently believed that, for some embodiments, the yield of formate in pure ethanol is less than in azeotrope ethanol as a result of the viscosity. Thus, azeotrope ethanol is an effective solvent as AMP can easily dissolved in azeotrope ethanol and it can be obtained from distilled bio-ethanol. Adding ethanol also can reduce the formation of stable bicarbonate and redirected CO₂ capture reaction towards other active carbon-containing species, without the loss of absorption efficiency.

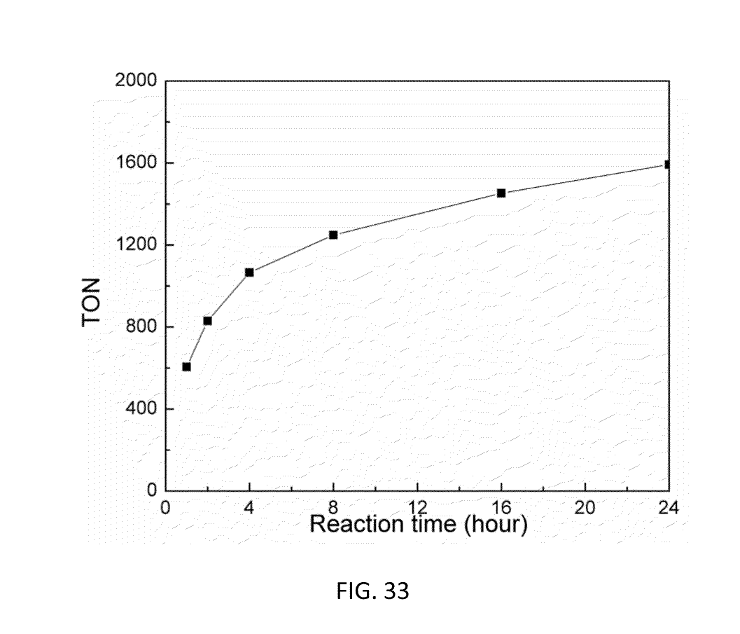

Solvent also can affect capture rate. To illustrate this, alcohol was added to water, and it was determined that adding ethanol can positively influence the performance of CO₂ capture and hydrogenation. Note that the final capacity for all proportion evaluated were same (0.96 mole CO₂ per mole AMP), but the capture rate was different. In one embodiment, the capture rate was gained at 70 wt % ethanol co-solvent (FIG. 33).

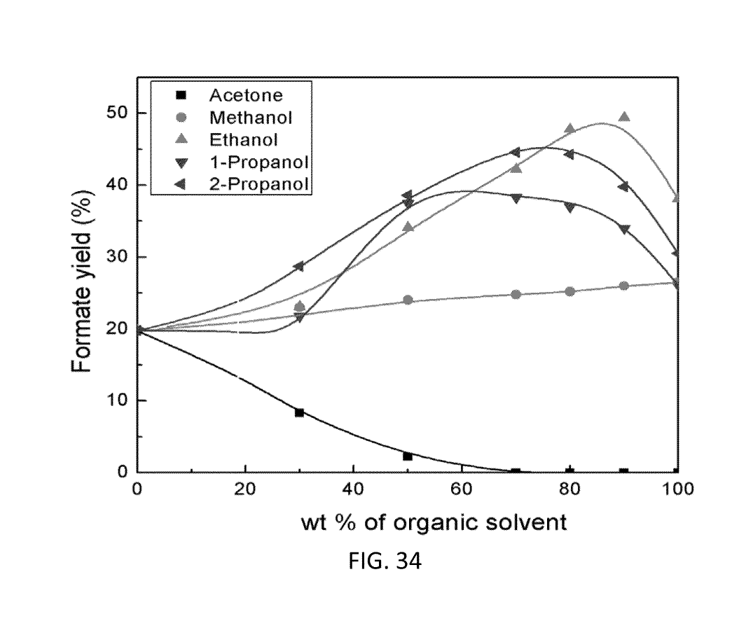

In yet additional embodiments, the pKa of the solvent used can influence the hydrogenation reaction of carbonate species, such as alkyl carbonate. As shown in FIG. 34, the hydrogenation efficiency observed for particular embodiments was in order of 2-Propanol>Ethanol=1-Propanol>>Methanol. Based on these results, it is currently believed that a higher pKa solvent favors the alkyl carbonate hydrogenation reaction. The increase in formate yield for 2-propanol, 1-propanol, and ethanol solvents may be due to their higher pKas (higher than that of water). The formate yield in embodiments using methanol as co-solvent remained almost unchanged, which may be explained by the fact that the pKa of methanol is lower that water.

A proposed mechanism accounting for carbamate (A), ethyl carbonate (B), and bicarbonate (C) formation is provided below.

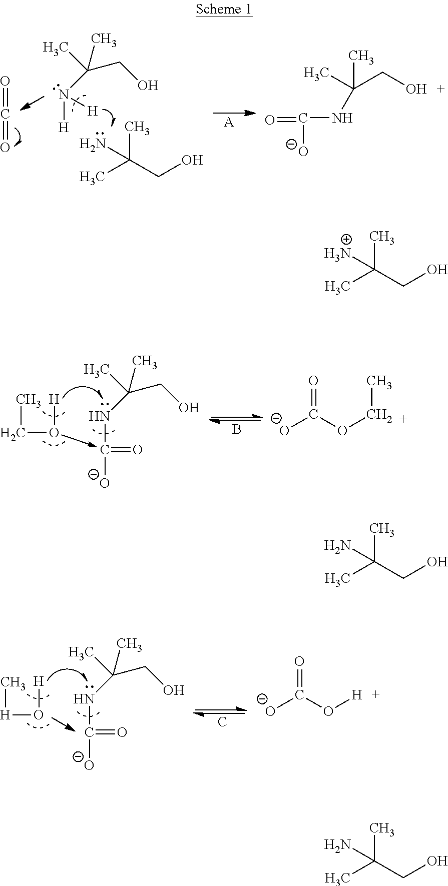

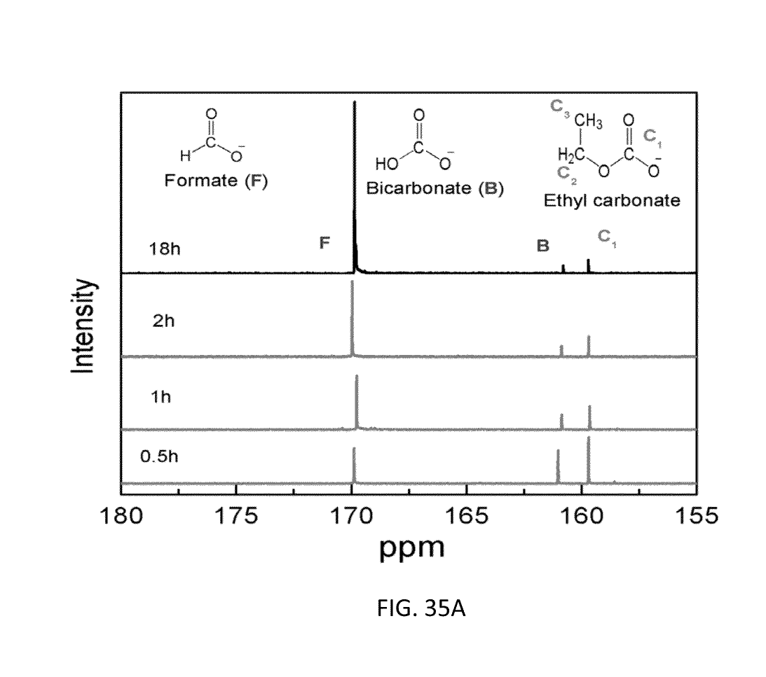

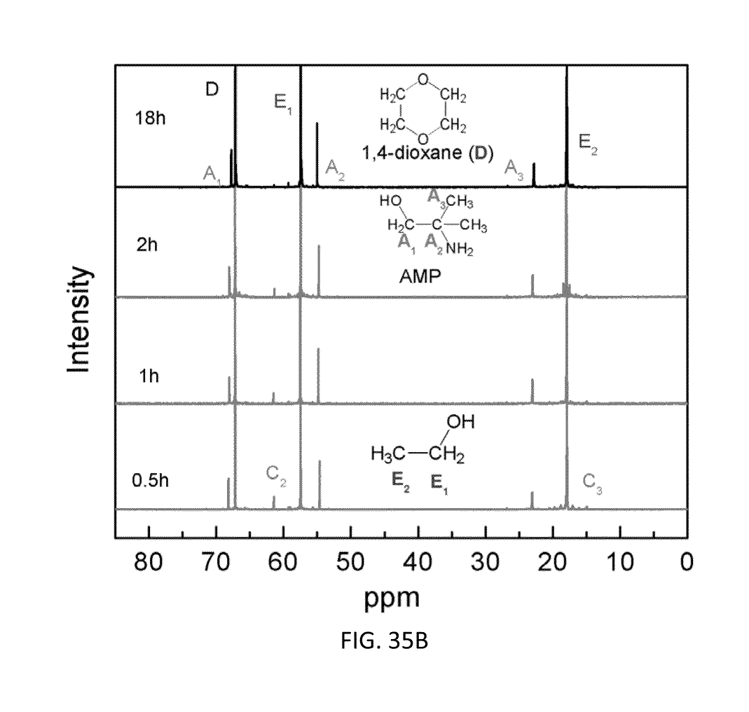

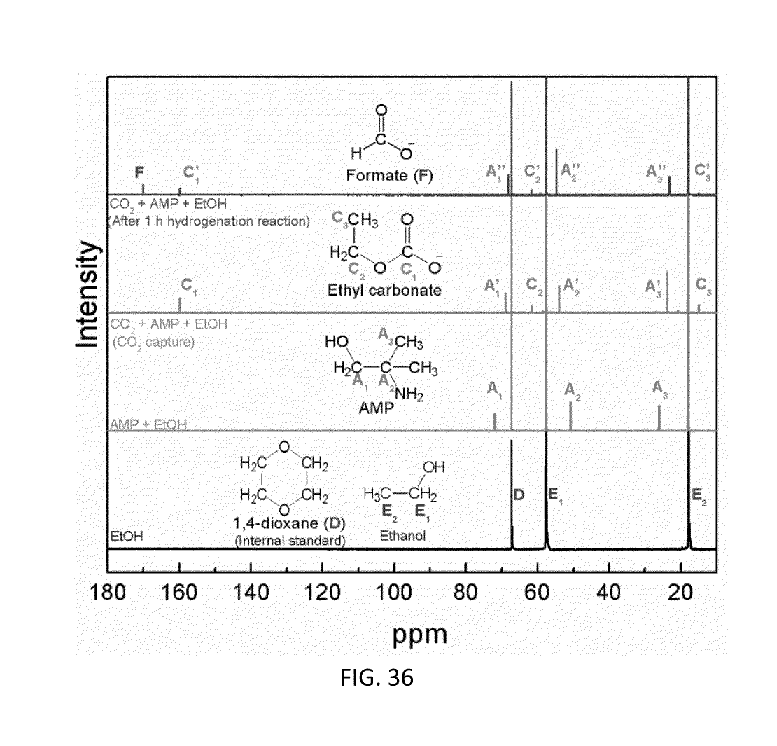

With reference to the proposed mechanisms in Scheme 1 and further in reference to FIGS. 35A and 35B, AMP-captured CO₂ produces ethyl carbonate, and after 0.5 hour hydrogenation reaction a peak located at 170 ppm, which belongs to formate. More formate (peak F in FIG. 35A) was produced while both bicarbonate (peak B in FIG. 35A) and ethyl carbonate (peaks C₁, C₂ and C₃ in FIG. 35A) were decreased with different hydrogenation reaction time from 0.5 hours to 18 hours. These spectra provide direct evidence establishing that both ethyl carbonate and bicarbonate are able to be hydrogenated to formate over the Pd/AC catalyst. FIG. 36 also gives a clear comparison of species before/after capture and hydrogenation reaction. The selectivity of the hydrogenation of amine-captured CO₂ was 100% in these examples as the formate was the only detected product after the reaction.

In the below-described examples, a kinetic study of bicarbonate in pure water and ethyl carbonate in pure ethanol was performed.

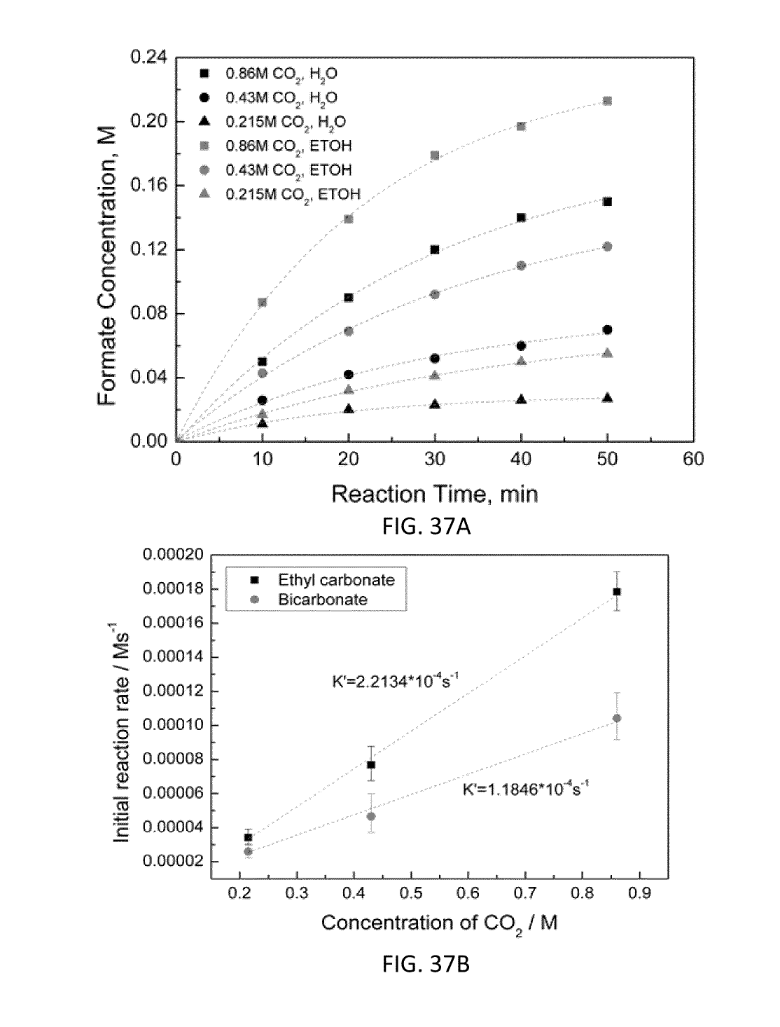

As shown in FIGS. 37A and 37B, the kinetics of the formation of formate through both bicarbonate and ethyl carbonate intermediates fits well with the first order kinetics. At the same time for both routes with different reaction intermediates, the conversion of formate increases with the increasing of adopted initial carbon dioxide concentration. With larger slopes of the rate curves at different reaction time, the reaction rates are also higher in experimental groups with higher carbon dioxide starting concentrations. By conducting the exponential fitting to the rate curves, the initial rate of formate formation/carbon dioxide consumption can be found conveniently. The initial rate of carbon dioxide consumption can be plotted as a function of initial concentration of captured carbon dioxide. Interestingly, there is a linear relation between initial carbon dioxide concentration and initial reaction rate, which indicates the hydrogenation of CO₂ is a pseudo-first order reaction with respect to the concentration of carbon dioxide. By taking hydrogen pressure into account, the overall expression of this pseudo-first order reaction can be elaborated into following form:

R=k_{eff}[CO₂]=f(kₛ,k_{L},kᵣ)[CO₂][H₂]

wherein k_{eff} is the overall effective reaction constant of the system, kₛ is the liquid-solid mass transfer coefficient, k_{L} is the gas-liquid mass transfer coefficient and kᵣ is the intuitive reaction constant of hydrogenation. The effective rate constant of hydrogenation of ethyl carbonate (2.2*10⁻⁴ s⁻¹) is about twice as large as that of the bicarbonate route (1.1*10⁻⁴ s⁻¹), indicating a larger activity of hydrogenation reaction in ethanol solvent for some embodiments.

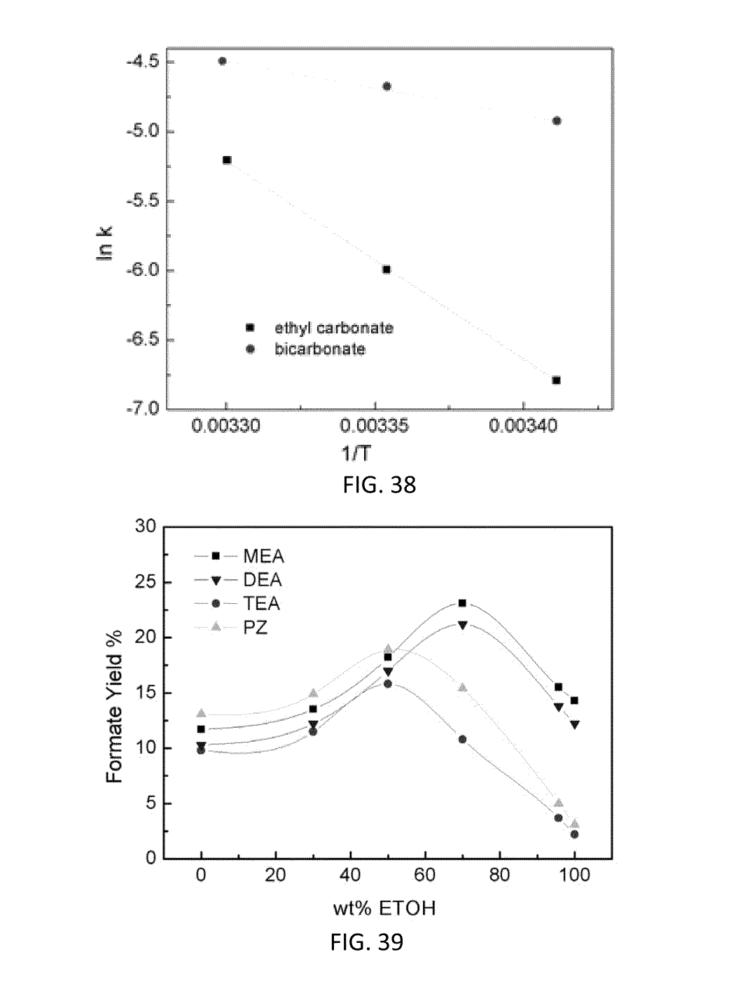

The activation energy was determined by using the Arrhenius equation (k=Aexp⁻Eₐ/RT). FIG. 38, which is a plot of ln k₁ vs 1/T, provides graphical kinetic data at different temperatures. In the temperature range of 20 to 40° C., the Eₐ for the reaction of bicarbonate to formate in water was 31.9 (kJ/mol), while for the reaction of ethyl carbonate to formate in 100% ethanol was 118.9 (kJ/mol). Although the observation kinetic rate of hydrogenation of bicarbonate is higher than ethyl carbonate, and the temperature sensitivity of the hydrogenation reaction of ethyl carbonate is larger than bicarbonate as well, it is currently believed that these results may explain why the yield of formate increases with the ethanol concentration increasing from 0% to 95.7%. From another point of view, the activation energy of hydrogenation of bicarbonate is very low, while low Eₐ values (<42 kJ/mol) usually indicate diffusion-controlled processes. In a catalytic system, the activation energy is lowered by mass transfer resistance. The effect is caused by the low-order temperature dependence of the diffusion coefficient. Catalytic reactions under mass transfer control may accelerate only as quickly as diffusion, and the resulting activation energy is superficially lowered. These values indicate mass transfer effects may dominate the overall reaction kinetics. Therefore, continually increased concentration of ethanol may significantly increase the solubility of hydrogen in aqueous phase, which enhances the external mass transfer.

To compare effect of amine's various properties, such as structure, solubility, functional group containing, and other proprieties on CO₂ capture and conversion, five amine molecules were selected as model compounds: monoethanolamine (MEA), diethanolamine (DEA) and Triethanolamine (TEA), 2-amino-2-methyl-1-propanol (AMP) and Piperazine (PZ).

To compare the effect of an amine's various properties, such as structure, solubility, functional group containing, and other proprieties, on CO₂ capture and conversion, five amine molecules were selected as model compounds: monoethanolamine (MEA), diethanolamine (DEA) and Triethanolamine (TEA), 2-amino-2-methyl-1-propanol (AMP), and Piperazine (PZ). Without being limited to a single theory, it is currently believed that sterically hindered amines can be used to reduce the carbamate intermediate's stability; thus, this carbamate can undergo hydrolysis to form bicarbonate more readily and also release free amine molecules for further reaction with CO₂ and consequently enhance the CO₂ equilibrium loading capacity. FIG. 17 shows the initial captured CO₂ rate for the various amines tested in this example before 15 minutes. As illustrated by FIG. 19, the order of reactivity of the tested amines was as follows (listed in order of decreasing reactivity): PZ>MEA>AMP>DEA>TEA. The final CO₂ capture amount that was observed exhibited the following order (listed in order of decreasing reactivity): PZ>AMP>DEA>MEA>TEA.

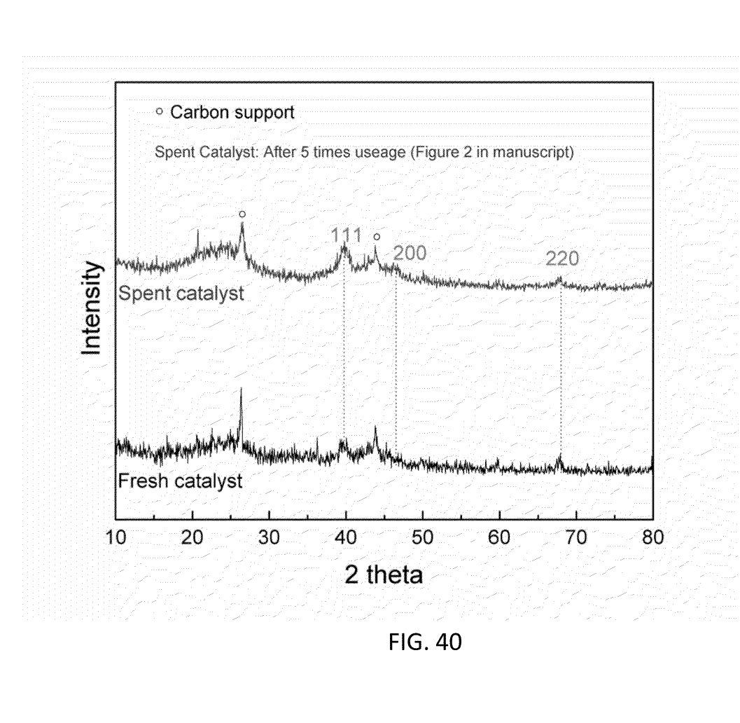

After capture of CO₂ with different amines, the hydrogenation reaction of captured CO₂ was examined. As illustrated in Table 6 and FIG. 39, the formate yield and the turn over number (TON) were determined. The order of reactivity observed was in the following order of increasing reactivity: PZ<TEA<DEA<MEA<AMP. This order of reactivity likely corresponds to the bicarbonate concentration produced by these amines. For MEA, DEA, and TEA, the formate yield was at the same level due to their similar bicarbonate concentration. AMP exhibited a high bicarbonate concentration (0.75M), which was almost two times that of MEA, DEA, and TEA. Accordingly, the formate yield and TON of AMP was also two times as high. PZ had the lowest formate yield (5.5%) even though it exhibited a high concentration of carbamate (0.69 M).

¹³C-NMR spectroscopy at room temperature has been proven to be a simple and reliable method to investigate the specification in solution of these carbon containing salts. CO₂ that has been captured with an amine could generate four different kinds of carbon species: bicarbonates, carbonates, carbamates, and carbamic acid. FIG. 20 is a ¹³C NMR spectrum illustrating the specification in solution of CO₂ captured by MEA, DEA, TEA, PZ, and AMP. The peaks around 161 ppm (indicated with “*”) were assigned to bicarbonate/carbonate, and the small peaks around 162 ppm to 165 ppm (indicated with “̂”) were assigned to carbamate. The MEA, DEA, and PZ exhibited of peaks of bicarbonate/carbonate and carbamate, whereas TEA and AMP only exhibited one peak, belong to bicarbonate/carbonate. AMP produced a high amount of bicarbonate (about 0.75M in some embodiments and about 0.93M in some other embodiments), thereby supporting the results establishing that AMP-captured CO₂ exhibits the best hydrogenation activity among the different amines tested in this example. In some embodiments, the chemical structures of different amines can explain why different amines have a different ion distribution. The carbamate species formed between AMP and CO₂ experiences an increased steric hindrance effect as compared to less-sterically hindered amines; therefore, this carbamate can more readily be decomposed in water. For the tertiary amine, TEA, the carbamate intermediate is not readily formed. Additionally, the cyclic nature of PZ may contribute to its ability to produce a more stable carbamate species and thereby exhibit a lower yield of bicarbonate and ultimately a lower formic acid yield than other amine species. In some embodiments, a cyclic amine like PZ can be added to increase the capturing rate. Solely by way of example, PZ has a cyclic, diamine structure that may facilitate rapid formation of carbamates when it reacts with CO₂. PZ can also theoretically absorb two moles of CO₂ for every mole of amine. However, due to the formation of protonated PZ carbamate at high CO₂ loading, the actual mole ratio of CO₂ to PZ is 1 (FIG. 40)

TABLE 8
The result of CO₂ capture and hydrogenation with different amine agent

| | | | | Captured CO₂ species concentration | | | | | | |
|---|---|---|---|---|---|---|---|---|---|---|
| | Adsorption results^{[a]} | | | (M)^{[d]} | | | | | | |
| | | | CO₂ | | | | NCO₂H | | Conversion results | |
| | | Solvent | Cap. | | | RNCO₂⁻ | Carbamic | Ethyl | Formate | |
| Entry | Amine | (wt % )^{[b]} | (%)^{[c]} | HCO₃⁻ | CO₃²⁻ | Carbamate | acid | carbonate | Yield (%) | TON^{[e]} |
| 1 | MEA | 0 | 0.77 | 0.67 | 0.07 | 0.03 | 0 | 0 | 11.7 | 167 |
| 2 | DEA | 0 | 0.82 | 0.65 | 0.16 | 0.01 | 0 | 0 | 10.3 | 147 |
| 3 | TEA | 0 | 0.59 | 0.58 | 0.06 | 0.00 | 0 | 0 | 9.7 | 115 |
| 4 | PZ | 0 | 0.98 | 0.28 | 0.03 | 0.69 | 0 | 0 | 13.1 | 230 |
| 5 | AMP | 0 | 0.96 | 0.84 | 0.12 | 0.00 | 0 | 0 | 19.8 | 316 |
| 6 | AMP | 30 | 0.96 | 0.90 | 0.01 | 0.01 | 0.04 | — | 23.1 | 365 |
| 7 | AMP | 50 | 0.96 | 0.73 | 0.01 | 0.02 | 0.21 | — | 34.1 | 538 |
| 8 | AMP | 60 | 0.96 | 0.45 | 0.00 | 0.03 | 0.48 | — | 35.7 | 564 |
| 9 | AMP | 70 | 0.96 | 0.32 | 0.00 | 0.03 | 0.60 | — | 42.2 | 666 |
| 10 | AMP | 80 | 0.96 | 0.18 | 0.00 | 0.03 | 0.74 | — | 33.8 | 534 |
| 11 | AMP | 100 | 0.96 | 0.00 | 0.00 | 0.03 | 0.93 | — | 24.1 | 380 |

^{[a]}CO₂ capture conditions: 20 ml amine/water solution, concentration of amine is 1M, capture temperature is 20° C., capture time is 40 min.
^{[b]}The concentration of different captured CO₂ species was got from NMR spectra.
^{[c]}Reaction conditions: after capture CO₂, directly transmit the 20 ml amine/water solution in to Parr reactor. Catalyst Pd Nps on carbon (5 wt %) is 0.1 g, reaction temperature is 20° C., Hydrogen pressure is 400 psi, reaction time is 1 hour.
^{[e]}TON was calculated by the formula: [total mole number of formate formed/(total mole number of Pd atom × 23.2%)] the 23.2% is the dispersion of Pd atom on the surface of Pd NPs, this result is come from the carbon monoxide chemisorption.

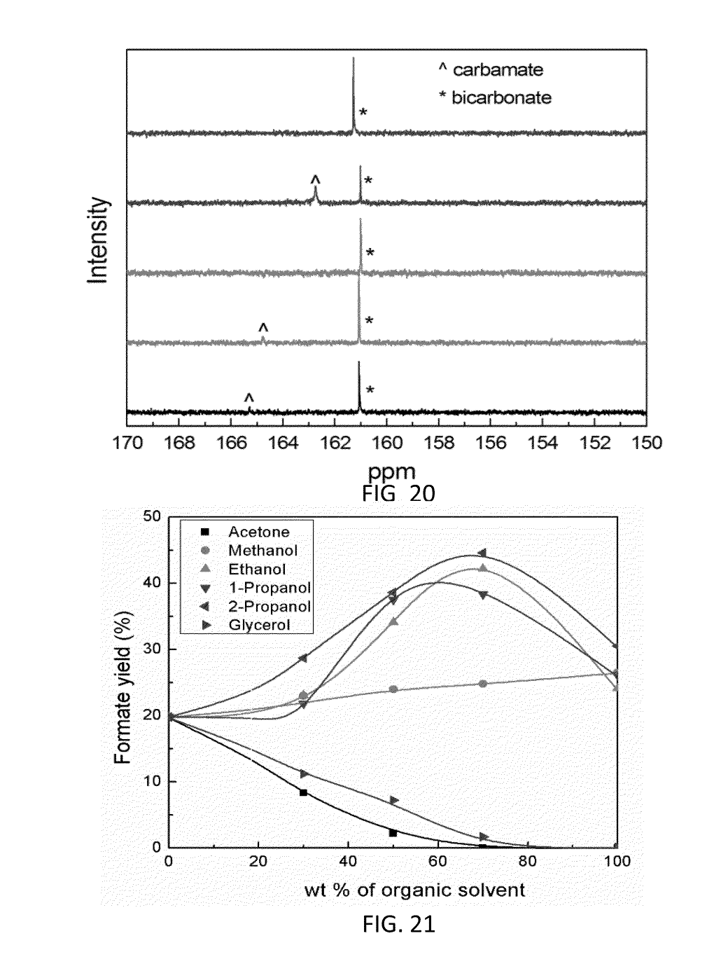

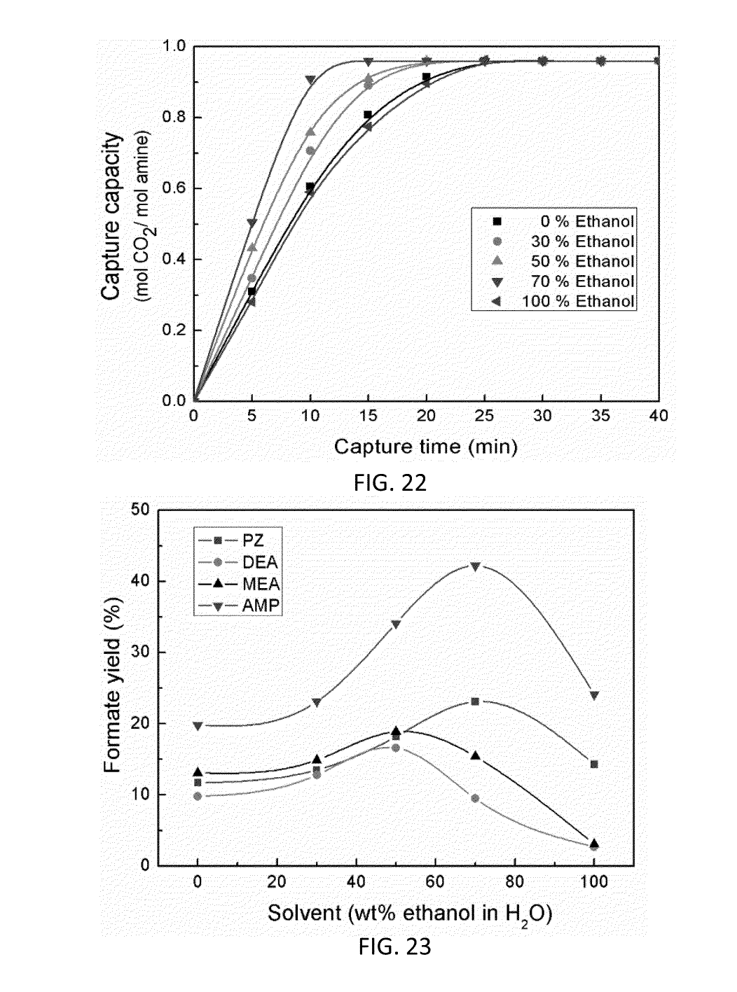

To further investigate solvent effects on the hydrogenation of different carbon containing salts in aqueous solutions, ¹³C-NMR spectroscopy at room temperature was used to identify the species in the solutions. As discussed below, the proportion of co-solvent can affect the capture rate and hydrogenation efficiency of amine-captured CO₂. In some embodiments it was determined that the solvent had a great effect on the performance of hydrogenation. In one example, the yield of formate was 19.8% and the TON reached 315.7 with the activated carbon supported palladium catalyst system (5% Pd/AC) after reacting for 1 hour in water at 20° C. (Table 8, entry 5). As shown in Table 8 and FIG. 21, AMP-captured CO₂ in anhydrous alcohol and various alcohol-water solutions illustrated different hydrogenation behaviours. In the co-solvent, methanol did not have a significant effect on formate formation, producing a formate yield around 20% to 25%. However, introducing 1-propanol, 2-proponal, or ethanol as the co-solvent increased the formate production, particularly when the proportion of 1-propanol, 2-proponal and ethanol was 70 wt %. In these examples, the formate yield was increased to 38.3%, 41.6% and 42.2%, respectively. But when using these anhydrous alcohols as solvent, the formate yield decreased. For example, the formate yield of 100 wt % ethanol solvent was 24.1% (Table 8, entry 11). Acetone and glycerol also were explored as co-solvents, but they appeared to quench the hydrogenation reaction. When using 100 wt % acetone or glycerol as solvent, there is almost no hydrogenation reaction. These results (also summarized in Table 8 and FIG. 22) corroborate that the hydrogenation activity of particular catalyst systems disclosed herein can be increased using amine capture in a co-solvent system. Although the final capacity for all proportion tested are same (0.96 mole CO₂ per mole AMP), the faster capture rate was gained 70 wt ethanol co-solvent. Additionally, use of this co-solvent system may help increase the solubility of the carbamate intermediate thereby also contributing to a higher observed activity.

On the other hand, as discussed above, organic solvents, such as ethanol, 1-propanol and 2-propanol, have a great improvement effect on the hydrogenation of amine-captured CO₂. FIG. 23 (Table 8, entry 1 to 5; Table 8, entry 6 to 12) shows the results of hydrogenation of different amine-captured CO₂ in different proportion of ethanol and water solution as co-solvent. PZ, MEA and DEA show the same trend with AMP. For PZ, the best formate yield (23.1%) was gained when the proportion of ethanol was 70 wt %, while, for MEA and DEA the maximum value of formate yield were gained when the proportion of ethanol was 50 wt %. Interestingly, in 100 wt % ethanol, formate yield with AMP and PZ as the capture agent was decreased as compared to the 70 wt % ethanol solvent, but a little higher than 100 wt % water system. In contrast to AMP and PZ, the formate yield with MEA and DEA as the capture agent decreased to <5%, which was much lower than the formate yield in water. Thus, although amines have different structure and chemical equilibrium mechanism, the co-solvent effect can take a more significant role to the reaction

As shown in FIG. 24, there is only peak located at 161.2 ppm which belongs to bicarbonate/carbonate ion pair. Interestingly, the introduction of ethanol as co-solvent created a new group of ¹³C-NMR peaks located at 160.5 to 159.8 ppm, indicating that a new carbon species form captured CO₂ was created. The formate yield in pure ethanol was 24.1%, which indicates that this new species also could be hydrogenated. Also in this example, different catalyst systems were explored in combination with using AMP to capture the CO₂. As illustrated in Table 9, high yields were obtained using Pd as the metal component and an activated carbon material as the solid support material. Without being limited to a single theory, it is currently believed that this observed reactivity may result from H₂ spillover only on the Pd surface, which can be influenced by H₂ dissociation to H⁺+e⁻ on the catalyst surface, transfer of electronic charge through the contact between the palladium and support particles, and/or electronic and protonic conductance. Also, the activated carbon support material can absorb CO₂ and H₂ effectively, thereby placing these species near the active sites on the Pd and contributing to an increased reactivity.

TABLE 9
Different catalysts comparison

Results

| Entry | Amine | Catalyst | Yield (%) | TON |
|---|---|---|---|---|
| 1 | AMP | Pd/Al₂O₃ | 8.61 | 135.9 |
| 2 | AMP | Pt/CaCO₃ | 0 | 0 |
| 3 | AMP | Pt/AC | 0 | 0 |
| 4 | AMP | Ru/C | 0 | 0 |
| 5 | AMP | Rh/C | 0 | 0 |
| 6 | AMP | Pd/AC | 42.2 | 666.1 |

### Example 3

### Ammonium Carbamate Reduction—

The low temperature Ammonium carbamate reduction reaction experiments were carried out in the 50 mL stirred Parr micro-reactor. The appropriate amounts of ammonium carbamate and catalyst system were added into 20 mL water. The reactor was then sealed, purged with high purity nitrogen three times, and then charged with the H₂ to the set pressure. During the reaction, mixing was achieved through an internal propeller operating at 620 RPM. Once the set temperature was attained, the reactor was held at the set temperature for a certain period of time and then quenched in an ice bath to quickly lower the temperature. The reactor was cooled until approximately 20° C., and then the gas pressure was recorded and vented. The reactor was immediately broken down and the liquid was collected for analysis.

The standard reaction conditions are: solvent is 20 ml, the concentrations of carbamate are 0.5 M, reaction temperature is 20° C., reaction pressure is 400 psi (H₂), and catalyst system loading is 0.1 g, reaction time is 1 hour.

Catalyst System Stability Testing (for Bicarbonate Reduction)—

The catalyst system stability testing was also carried out in the same Parr micro-reactor. The fresh catalyst system after one time reaction was separated by a centrifugation and washed with water and ethanol for 5 times, then the catalyst system was dry at 50° C. in N₂ for 6 hours. At last the spent catalyst system was reused in the carbamate reduction reaction. And the typical reaction conditions were: 20 ml solvent (70 wt % ethanol in water), 0.5 M ammonium carbamate, 20° C. reaction temperature, 2.75 MPa initial H₂ pressure, 0.1 g 5% Pd/AC catalyst, and 1 hour reaction time.

Aqueous-Phase Product Analysis—

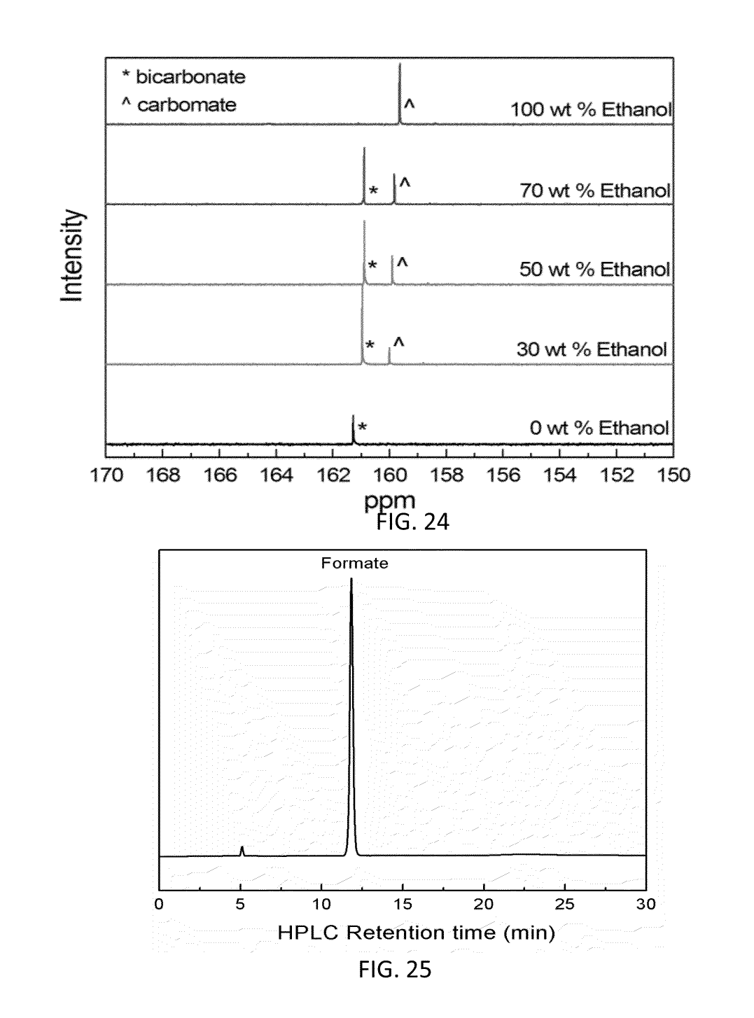

HPLC analysis was performed using a Shimadzu HPLC system equipped with a dual UV-VIS Detector (Shimadzu SPD 10-AV) at 208 and 290 nm and a Refractive Index Detector (Shimadzu RID-10A). For analysis of organic acids and reaction intermediates, the samples were separated in an Aminex 87-H column from Bio-Rad, using 5 mM H₂SO₄ as the mobile phase at 0.7 mL/min flow and a column temperature of 55° C. All samples for ESI-MS analysis were diluted with a base solution containing 0.1 wt % triethylamine and the analysis was performed using a Waters Micromass ZQ quadrupole mass spectrometer. An exemplary HPLC is provided in FIG. 25.

Pulse Chemisorption on Pd/AC Sample—

The analyzer was Micromeritics Autochem II 2920 unit. Before the test the sample was pretreated by heating under inert flow Helium (50 mL/min) at 250-350° C. for 60 minutes to remove adsorbed moisture. Then the sample was reduced by 10% H₂ in Ar at 250° C. for 1 hour, followed by helium purge at the same temperature for another 1 hour to remove the physical absorbed H₂ on the surface of the catalyst. CO-pulse chemisorption experiment was carried out at 40° C. using Helium gas with flow rate of 50 mL/min as carrier gas. With recording (0.2 seconds), the defined amount (0.5 mL) of (10% CO in He) was pulsed to the reactor in Helium carrier gas. The above step was repeated until desorption peaks reached the saturation value. The pulsation was terminated when two consecutive CO peaks resulted in an equal amount of CO observed according to the peak area. Between the pulses, the reactor was kept under 50 mL/min Helium flow. (The specific operations were performed following the manual of Micromeritics Autochem II 2920 analyzer) NMR—NMR measurements were performed on a 2-channel 400 MHz Varian VNMRS with an ATB automation probe (1000 scan, decouple, a pulse width of 90° and a recovery delay of 25 seconds). The sample preparation method is as follows: add 0.005 mole carbon salts in 10 ml solvent in a vial, and add 0.04 ml 1,4-Dioxane (67.19 ppm) as internal standard; then put these sealed vials into ultrasonic washer (10 minutes) for accelerate the dissolution; at last, certain amount of clear liquid was put into NMR tube for analysis.

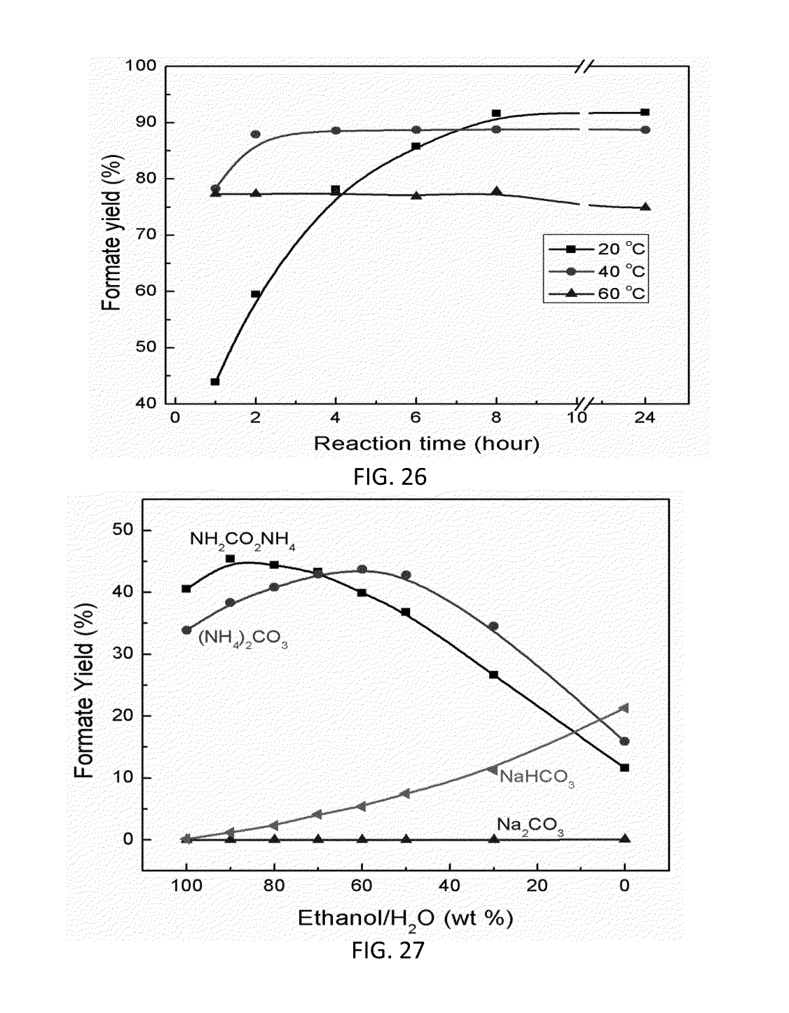

Catalytic hydrogenation of ammonium carbamate is described in this example. Table 10 and FIG. 26 show the results of catalytic hydrogenation of ammonium carbamate in anhydrous ethanol and various ethanol-water solutions. The yield of formate was 40.5% and the TON reached 373.3 using the activated carbon supported palladium catalyst system embodiment (5% Pd/AC) after reaction in anhydrous ethanol for 1 hour at 20° C. (Table 10, entry 1). To increase the solubility of ammonium carbamate, water was added as a co-solvent to the ethanol-based embodiments. Using this co-solvent system, the formate yield reached 43.9% (Table 10, entry 2). The yield and TON were further increased by allowing the reaction to continue for a longer time period (e.g., 8 hours). These examples illustrate that the hydrogenation of ammonium carbamate in ethanol/water solutions is very rapid with H₂ and particular catalyst system embodiments, such as the Pd/AC catalyst system. It was further observed that increasing the proportion of water decreased the formate yields in certain embodiments (e.g., Table 10 entries 3 to 5).

As shown in Table 10, other influence factors such as H₂ pressure and reaction temperature also can affect the reactivity of the hydrogenation reaction. In some embodiments, the yields of formates could be improved by increasing the H₂ pressure, possibly increasing the solubility of H₂ in the solvent (Table 10, entry 1 and 11 to 14). Also, by increasing the reaction temperature to 40° C., the formate yield could be increased (e.g., increased to 87.9%, as with the case of entry 9 in Table 10). As shown in FIG. 26, thermodynamic equilibria for the hydrogenation of ammonium carbamate can exist in a batch process at different temperatures. In some embodiments, higher reaction temperatures can be used to improve initial formate yields; however, in some embodiments higher reaction temperatures can ultimately decrease the equilibrium yields of formate.

TABLE 10
Catalytic hydrogenation of ammonium carbamate

| | Reaction conditions | | | | | |
|---|---|---|---|---|---|---|
| | Solvent^{[b]} | | Pressure | Time | Yield | |
| Entry | (wt %) | Temperature | of H₂ (MPa) | (h) | (%) | TON^{[c]} |
| 1 | 100 | 20 | 2.75 | 1 | 40.5 | 373.3 |
| 2 | 70 | 20 | 2.75 | 1 | 43.9 | 404.6 |
| 3 | 50 | 20 | 2.75 | 1 | 24.8 | 228.6 |
| 4 | 30 | 20 | 2.75 | 1 | 17.6 | 162.2 |
| 5 | 0 | 20 | 2.75 | 1 | 11.6 | 106.9 |
| 6 | 70 | 20 | 2.75 | 2 | 59.5 | 548.4 |
| 7 | 70 | 20 | 2.75 | 6 | 85.8 | 790.8 |
| 8 | 70 | 20 | 2.75 | 8 | 91.7 | 845.2 |
| 9 | 70 | 40 | 2.75 | 2 | 87.9 | 810.2 |
| 10 | 70 | 60 | 2.75 | 2 | 77.3 | 712.5 |
| 11 | 70 | 20 | 0.69 | 1 | 28.5 | 262.7 |
| 12 | 70 | 20 | 1.38 | 1 | 42.3 | 389.9 |
| 13 | 70 | 20 | 4.14 | 1 | 47.6 | 438.1 |
| 14 | 70 | 20 | 5.52 | 1 | 52.4 | 482.0 |

^{[a]}Reaction conditions: 20 ml solvent, concentration of ammonium carbamate is 0.5M, catalyst system is 0.1 g, reaction temperature is 20 to 60° C. Catalyst Pd nano-particles on active carbon (Pd/AC) were 5% Pd loading and purchased form Sigma-Aldrich.
^{[b]}Reaction solvent is ethanol-water solution, 100 wt % means anhydrous ethanol.
^{[c]}TON was calculated by the formula: total mole number of formatted formed/(total mole number of Pd atom × 23.2%) wherein the 23.2% is the dispersion of Pd atom on the surface of Pd NPs, this result is come from the carbon monoxide chemisorption

In this example, the efficiency of the hydrogenation reaction of ammonium carbamate as compared with other carbon containing salts such as NaHCO₃, Na₂CO₃ etc., was determined. As shown in FIG. 27, hydrogenation efficiency of NH₂CO₂NH₄, NH₄HCO₃, (NH₄)₂CO₃, NaHCO₃ and Na₂CO₃ were compared using different ratios of ethanol-water mixed solvents at room temperature. In embodiments using only water as the solvent, the hydrogenation of NaHCO₃ was faster than hydrogenation of NH₂CO₂NH₄ and (NH₄)₂CO₃. In contrast, the hydrogenation of NaHCO₃ was completely suppressed in the anhydrous ethanol solution, whereas NH₂CO₂NH₄ and (NH₄)₂CO₃ reactions were still active. When an ethanol (70 wt %)/water co-solvent system was used, the formate yields from the hydrogenation of either NH₂CO₂NH₄ or (NH₄)₂CO₃ reached approximately 43%. In contrast, the formate yield obtained from the hydrogenation of NaHCO₃ was only 4.0% in ethanol/water co-solvent system, which suggests that the hydrogenation efficiency of NH₂CO₂NH₄ and (NH₄)₂CO₃ is at least 10 times higher than that of NaHCO₃ under the same reaction conditions. In some embodiments, (NH₄)₂CO₃ and Na₂CO₃ showed completely different hydrogenation activities. In some embodiments, Na₂CO₃ experienced little to no hydrogenation, while (NH₄)₂CO₃ was hydrogenated easily, and exhibited a similar hydrogenation efficiency trend as NH₂CO₂NH₄.

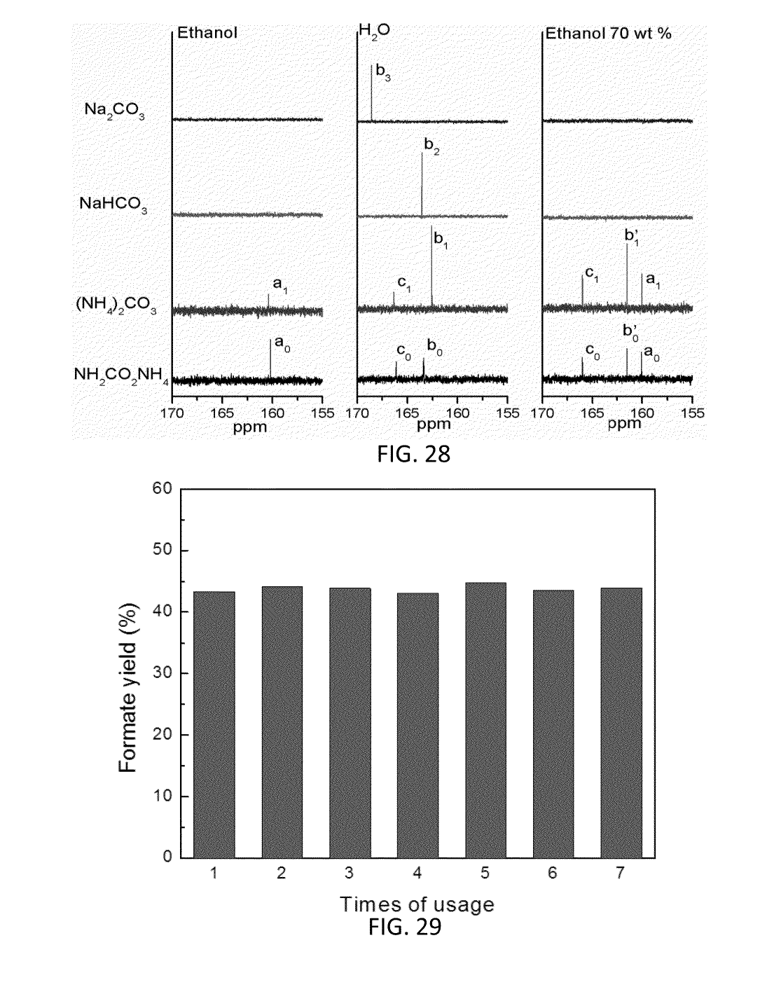

In additional examples, the solvent effects on the hydrogenation of different carbon containing salts in aqueous solutions were determined using ¹³C-NMR spectroscopy at room temperature. This characterization method was used to identify the species in the solutions. FIG. 28 shows the ¹³C-NMR spectra of the solutions of NH₂CO₂NH₄, (NH₄)₂CO₃, NaHCO₃, and Na₂CO₃ in different solvents, revealing four different groups of signals. In the mixed ethanol/water solvents or anhydrous ethanol, new signals a₀ and a₁ (both peaks located at 160.2 ppm) were displayed and assigned to carbamic acid. The formation of carbamic acid, which holds the C═O functionality at δ(C═O) around 160 ppm, is usually obtained by reacting CO₂ with ammonia or amine in organic solvent. These peaks may also confirm that ammonium carbamate is in equilibrium with carbamic acid (NH₂CO₂H) and NH₃ (Equation 2) in non-polar organic solvent.

NH_{z}CO_{z}NH₄ [symbol] NH_{z}CO_{z}H+NH₂ (2)

Signals c₀ (166.3 ppm) and c₁ (166.2 ppm) may be assigned to carbamate ions. Signals b₀ (163.4 ppm), b₁ (162.6 ppm), b₂ (163.5 ppm) and b₃ (168.5 ppm) likely are due to the carbonate/bicarbonate ion pair with fast proton exchange. And signals b′₀ and b′₁ (both peaks located at 161.5 ppm) may originate from the carbonate/bicarbonate ions, which are shifted due to the solvent effect. The results summarized in Table 10 and FIGS. 26 and 27 indicate that dissolved NH₂CO₂NH₄ has a very high activity for hydrogenation. As shown in other examples disclosed herein, the signal belonging to the carbamate ion also could be detected in an aqueous solution of (NH₄)₂CO₃, thereby indicating that a portion of the carbonate ion could convert to the carbamate ion in presence of ammonium or ammonia. In some embodiments, NH₂CO₂NH₄ and (NH₄)₂CO₃ gave an almost identical spectrum, particularly in the presence of ethanol. This result likely indicates the reason for why the hydrogenation behaviour of (NH₄)₂CO₃ and NH₂CO₂NH₄ was similar. In contrast, due to the fact that the CO₃²⁻ ion of Na₂CO₃ cannot be hydrogenated, and therefore cannot be converted to a carbamate, this species has a lower hydrogenation yield. NaHCO₃ can exhibit good hydrogenation efficiency in water, however, when the proportion of ethanol in the solvent system is increased, the NaHCO₃ also cannot convert to a carbamate species and maintain solubility, thereby also exhibiting a lower formate yield in certain conditions. About 5% formate was produced from the hydrogenation of NaHCO₃ in the 70 wt % ethanol-water solutions; however, no ionic species were observed in the reactant solutions with ¹³C NMR. On potential explanation is that the concentration of bicarbonate in ethanol was too low to detect. The ¹³C NMR spectra of NH₂CO₂NH₄ and (NH₄)₂CO₃ in the 70 wt % ethanol-water solvent were almost identical, and in general the specification of these two ammonium salts are similar in pure water, ethanol, or other ethanol-water solutions, as seen in FIG. 28, which explains why the production of formate salts from the hydrogenation of (NH₄)₂CO₃ and NH₂CO₂NH₄ followed the similar trend with respect to the different ratio of ethanol to water of the solvents. Carbamic acid was found to be unstable in aqueous solutions, while its stability was improved in less polar ethanol and ethanol-water solvents.

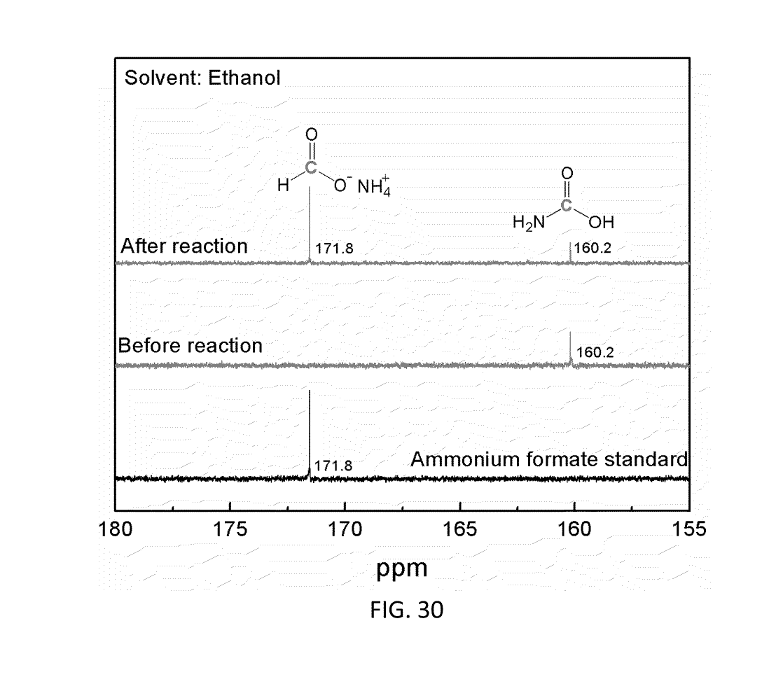

The methods disclosed in this example provide benefits, such as facile products separation and high catalyst system stability. Separation of ammonium formate products from ethanol or ethanol-water solvents is much easier than separating such products from water, given the low boiling point and low specific heat capacity of the solvents. Secondly, Pd/AC heterogeneous catalysts are easier to handle, separate, and recycle than homogeneous catalysts. Moreover, the Pd/AC catalyst system shows a high stability, as show in FIGS. 29 and 30, as even after 7 times repeated reactions without regeneration, the spent catalyst system had no activity loss compared to the fresh catalyst.

The feasibility of a new CO₂ reduction strategy with ammonia or amine-captured CO₂, in the form of ammonium carbamate and carbonate, as the feedstock for the hydrogenation reaction to produce formate has been developed. At the optimized reaction conditions, ˜92% formate yield was obtained from the hydrogenation of ammonium carbamate in the 70 wt % ethanol-water solution with 2.75 MPa H₂ and the Pd/AC catalyst system after reacting for 8 hours at 20° C. Carbamic acid was stable in pure ethanol and became less stable with increasing the water content in ethanol-water solvents. Both carbamic acid and carbamate ions are reactive with respect to the hydrogenation reaction to produce formate. The efficiency of hydrogenation of ammonium carbamate and carbonate was much higher than that of alkali metal bicarbonate in the presence of ethanol solvent, which may be due to the presence of active carbamic acid and carbamate ion species. Accordingly, new industrial CO₂ utilization methods can be developed with the disclosed catalyst systems and methods. The new strategies disclosed herein utilize the CO₂ intermediates from urea production or aqueous ammonia scrubbing CO₂ processes as feedstocks to produce value-added commodity formate chemicals, with the possibility of leveraging the existing industrial infrastructure for commercialization.

TABLE 11
Catalytic hydrogenation of ammonium carbamate
in ethanol over different catalysts.^{a}

| Entry | Catalyst | Yield (%) |
|---|---|---|
| 1 | Pd/AC | 40.5 |
| 2 | Ru/AC | 0.1 |
| 3 | Rh/AC | 0.4 |
| 4 | Pt/AC | 0.0 |
| 5 | Ni/AC | 0.0 |
| 6 | Pd/Al₂O₃ | 9.6 |
| 7 | Pd/CaCO₃ | 0.6 |
| 8 | Pd/BaSO₄ | 4.3 |

^{a}The reaction conditions are: solvent is (100 wt % ethanol) 20 ml, the concentration of carbamate is 0.5M, reaction temperature is 20° C., reaction pressure is 2.75 MPa (H₂), catalyst system loading is 0.1 g, and reaction time is 1 hour.

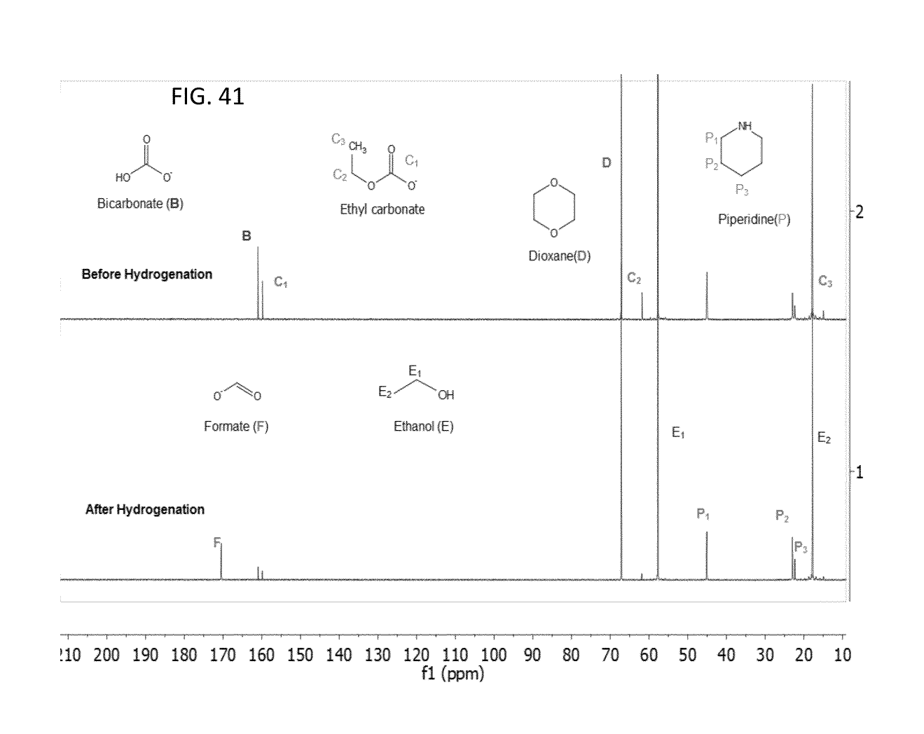

In some examples, reactions with piperidine were evaluated and this compound exhibited excellent hydrogenation activities. After one hour of the hydrogenation reaction, the formate yield reached 95.3% and the TON was 1465 (see Entry 6 in Table 12, below). Without being limited to a particular theory of operation, it is currently believed that the activity of piperidine may result because the pKa of piperidine is 13.4 while AMP is only 9.3 (the higher pKa may be at least one reason for the observed improvement to the hydrogenation reaction). Yet another potential reason for the activity observed for piperidine may be the effect of different intermediates. The NMR spectrum illustrated in FIG. 41 (taken after hydrogenation) illustrates that there is no carbamate remaining, indicating that piperidine-carbamate might hydrolyze to bicarbonate or undergo alcoholysis to produce ethylcarbonate since the more strongly basic properties of piperidine favor protonation of the secondary amine nitrogen as opposed to nucleophilic addition to the CO₂ carbon. Accordingly, it is currently believed that the initial carbamate product is fully hydrolyzed to bicarbonate or alcoholyzed to ethyl carbonate, such as is illustrated in the mechanistic Scheme 2 below.

TABLE 12

| | Solvent, wt % | Reaction | H₂ Pressure, | Reaction | FA | |
|---|---|---|---|---|---|---|
| Entry | ETOH | Temp, ° C. | psi | time, h | yield | TON |
| 1 | 0 | 20 | 400 | 1 | 50.2% | 768 |
| 2 | 50 | 20 | 400 | 1 | 78.0% | 1179 |
| 3 | 50 | 30 | 400 | 1 | 86.4% | 1317 |
| 4 | 50 | 30 | 400 | 1.5 | 96.6% | 1486 |
| 5 | 70 | 20 | 400 | 1 | 90.6% | 1394 |
| 6 | 70 | 30 | 400 | 1 | 95.3% | 1465 |
| 7 | 90 | 20 | 400 | 1 | 82.4% | 1267 |
| 8 | 95.7 | 20 | 400 | 1 | 68.6% | 1059 |
| 9 | 100 | 20 | 400 | 1 | 62.2% | 960 |

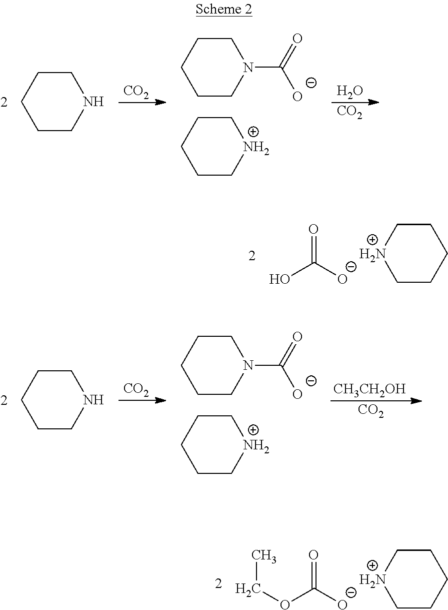

In view of the many possible embodiments to which the principles of the disclosed technology may be applied, it should be recognized that the illustrated embodiments are only preferred examples of the present disclosure and should not be taken as limiting the scope of the technology. Rather, the scope of the present disclosure is defined by the following claims. I therefore claim all that comes within the scope and spirit of these claims.

I claim:

1. A method for producing formate(s), formic acid, or a mixture thereof, from CO₂, comprising:
exposing a CO₂-derived compound other than sodium bicarbonate to a heterogeneous catalyst system comprising Pd and a carbon-based material; and
exposing the CO₂-derived compound to H₂ gas at a pressure ranging from 300 psi to 500 psi; wherein the CO₂-derived compound is exposed to the heterogeneous catalyst system and the H₂ gas at a temperature and for a time suitable to produce formate(s), formic acid, or a mixture thereof.

2. The method of claim 1, further comprising exposing CO₂ to an amine-containing compound to form the CO₂-derived compound.

3. The method of claim 2, wherein the amine-containing compound has a formula NH₂R^{a}, wherein R^{a} is aliphatic or heteroaliphatic; NH(R^{a})₂, wherein each R^{a} independently is aliphatic, heteroaliphatic, or wherein both R^{a} groups form, together with the nitrogen atom to which they are attached, a heterocyclic group comprising from 0 to 3 additional heteroatoms in addition to the nitrogen atom to which each R^{a} is attached; or N(R^{a})₃, wherein each R^{a} independently is aliphatic, heteroaliphatic, or wherein two or three R^{a} groups form, together with the nitrogen atom to which they are attached, a heterocyclic group comprising from 0 to 3 additional heteroatoms in addition to the nitrogen atom to which each R^{a} is attached.

4. The method of claim 2, wherein the amine-containing compound is selected from ammonia, monoethanolamine, diethanolamine, triethanolamine, 2-amino-2-methyl-1-propanol, N-methyldiethanolamine, N-methylethanolamine, 1,4-diaminobutane, 1,3-diamino-2-propanol, 2-(diethylamino)ethanol, 1,3-propanediamine, 2-diisopropylamino-ethanol, 2,2-dimethyl-1,3-propanediamine, N-1-methyl-1,3-propanediamine, N-tert-butyldiethanolamine, piperazine, piperidine, pyrrolidine, homopiperazine, 1-piperazineethanol, or combinations thereof.

5. The method of claim 1, wherein the CO₂-derived compound is exposed to a solvent selected from water, an alcohol, or a combination thereof.

6. The method of claim 5, wherein the solvent comprises water and 20 wt % to 90 wt % ethanol.

7. The method of claim 6, wherein the solvent comprises water and 95.6 wt % ethanol.

8. The method of claim 1, wherein the heterogeneous catalyst system comprises Pd nanoparticles supported on activated carbon.

9. The method of claim 1, wherein the CO₂-derived compound is exposed to H₂ at a pressure ranging from 350 psi to 450 psi, the temperature ranges from 20° C. to 80° C. and the CO₂-derived compound is exposed to the heterogeneous catalyst system and H₂ for a time period ranging from 20 minutes to 6 hours.

10. The method of claim 1, wherein the CO₂-derived compound has a formula (Z)₂CO₃, wherein each Z independently is selected from a metal, hydrogen, ammonium, or a quaternary ammonium group.

11. The method of claim 1, wherein the CO₂-derived compound is selected from potassium bicarbonate, ammonium bicarbonate, potassium carbonate, sodium carbonate, magnesium carbonate, calcium carbonate, ammonium carbonate, or a combination thereof.

12. The method of claim 2, wherein the CO₂-derived compound is an amine-captured CO₂-derived compound selected from ammonium carbamate, (1-hydroxy-2-methylpropan-2-yl)carbamate, or a mixture thereof.

13. The method of claim 1, wherein the CO₂-derived compound is first converted to a bicarbonate other than sodium bicarbonate or to a carbamate, and then to formate, formic acid, or a mixture thereof.

14. The method of claim 2, wherein the CO₂-derived compound is converted directly to formate, formic acid, or a mixture thereof.

15. The method of claim 1, wherein the CO₂-derived compound is ammonium bicarbonate, and the heterogeneous catalyst system comprises palladium on activated carbon.

16. A method of converting CO₂ to formate(s), formic acid, or a mixture thereof, comprising:
exposing CO₂ to an amine-containing compound to form a carbamate;
exposing the carbamate to a heterogeneous catalyst system comprising Pd and a carbon-based material; and
exposing the carbamate to H₂ gas at a pressure ranging from 300 psi to 500 psi;
wherein the carbamate is exposed to the heterogeneous catalyst system and the H₂ gas at a temperature and for a time suitable to produce formate(s), formic acid, or a mixture thereof.

17. A combination, comprising:
ammonium bicarbonate or a carbamate; and
a heterogeneous catalyst system comprising Pd and a carbon-based material.

18. The combination of claim 17, further comprising H₂ gas.

19. The combination of claim 17, further comprising an aqueous solvent, an alcohol, or a combination thereof.

20. The combination of claim 19, wherein the alcohol is selected from ethanol, methanol, 1-propanol, 2-propanol, butanol, isobutanol, pentanol, glycerol, or combinations thereof.

21. The combination of claim 17, wherein the carbamate is (1-hydroxy-2-methylpropan-2-yl)carbamate or ammonium carbamate.

22. The combination of claim 17, wherein the carbon-based material comprises one or more dopants selected from nitrogen, boron, oxygen, phosphorus, aluminum, phosphorus, tin, gallium, nickel, indium, and combinations thereof.
